(19) United States
(12) Patent Application Publication
(10) Pub. No.: US 2025/0204792 A1
(43) Pub. Date: Jun. 26, 2025

(54) HEMODYNAMIC MONITOR FOR TRIAGING PATIENTS WITH AORTIC STENOSIS

(71) Applicant: Becton, Dickinson and Company, Franklin Lakes, NJ (US)

(72) Inventors: Arghavan Arafati, Mission Viejo, CA (US); Cristhian M. Potes Blandon, Rancho Santa Margarita, CA (US); Feras Al Hatib, Huntington Beach, CA (US); Sai Prasad Buddi, Hyderabad (IN)

(21) Appl. No.: 19/080,399

(22) Filed: Mar. 14, 2025

Related U.S. Application Data
(63) Continuation of application No. PCT/US2023/032948, filed on Sep. 15, 2023.
(60) Provisional application No. 63/375,843, filed on Sep. 15, 2022.

(51) Int. Cl. A61B 5/02; A61B 5/00; A61B 5/022

(52) U.S. Cl. CPC A61B 5/02007; A61B 5/022; A61B 5/4842; A61B 5/7267; A61B 5/742; A61B 5/746

(57) ABSTRACT

A hemodynamic monitor for detecting aortic stenosis includes a non-invasive blood pressure sensor and an integrated hardware unit with a system processor, a system memory, and a display with a user interface. The system memory includes instructions that, when executed by the system processor, are configured to adjust, by a pressure controller, a pressure within an inflatable blood pressure bladder. An arterial pressure waveform data of the patient is generated based on the adjusted pressure within the inflatable blood pressure bladder over the period of time and a plurality of signal measures are extracted from the arterial pressure waveform data of the patient. Input features are extracted from the plurality of signal measures that are indicative of an aortic stenosis score of the patient, and the aortic stenosis score of the patient is determined based on the extracted input features.

### CROSS-REFERENCE TO RELATED APPLICATION(S)

This application is a continuation of PCT Application No. PCT/US2023/032948, filed Sep. 15, 2023, entitled “HEMODYNAMIC MONITOR FOR TRIAGING PATIENTS WITH AORTIC STENOSIS”, which claims the benefit of U.S. Provisional Application No. 63/375,843, filed Sep. 15, 2022, and entitled “HEMODYNAMIC MONITOR FOR TRIAGING PATIENTS WITH LOW EJECTION FRACTION OR AORTIC STENOSIS,” the disclosures of which are hereby incorporated by reference in their entireties.

### BACKGROUND

The present disclosure relates generally to aortic stenosis, and in particular to a system and method for detecting aortic stenosis in a patient.

Aortic stenosis is a condition where leaflets of an aortic valve become stiff, which causes a narrowing of an opening of the aortic valve. The aortic valve of a patient with aortic stenosis is unable to fully open and fully close like a healthy aortic valve. The narrowing of the aortic valve and the inability of the aortic valve to fully open and close reduces the flow of blood through the aortic valve into the systemic circulatory system. Traditionally, aortic stenosis is detected and measured in a patient through image tests, such as an echocardiogram, an electrocardiogram, a chest X-ray, a computerized tomography (CT) scan, or cardiac magnetic resonance imaging (MRI). Other tests used to detect and measure aortic stenosis include cardiac catheterization and nuclear stress testing. Each of these tests requires a highly-trained specialist to perform the test and interpret the results of the test. Thus, patients must travel to a cardiologist or other cardiovascular specialist to get an initial heart screening. These tests can be expensive and can possibly take days or weeks to inform the patient if they have aortic stenosis and the level of aortic stenosis. A solution is needed that will provide greater access to aortic stenosis screening for patients with less travel. Preferably, the solution will also reduce the amount of time patients must wait to get results from aortic stenosis screenings so patients can seek further testing and/or treatment with less delay.

### SUMMARY

In one example, a hemodynamic monitor for detecting aortic stenosis is disclosed. The hemodynamic monitor includes a non-invasive blood pressure sensor with an inflatable blood pressure bladder, a pressure controller pneumatically connected to the inflatable blood pressure bladder, and an optical transmitter and an optical receiver that are electrically connected to the pressure controller. The hemodynamic monitor further includes an integrated hardware unit with a system processor, a system memory, and a display with a user interface. The system memory includes instructions that, when executed by the system processor, are configured to adjust, by the pressure controller, a pressure within the inflatable blood pressure bladder to maintain a constant volume of an artery of a patient for a period of time based on a feedback signal generated by the optical transmitter and the optical receiver. The instructions, when executed by the system processor, are further configured to generate an arterial pressure waveform data of the patient based on the adjusted pressure within the inflatable blood pressure bladder over the period of time and extract a plurality of signal measures from the arterial pressure waveform data of the patient. The instructions, when executed by the system processor, are further configured to extract input features from the plurality of signal measures that are indicative of an aortic stenosis score of the patient; determine the aortic stenosis score of the patient based on the extracted input features; generate a first sensory alarm signal configured to generate a first sensory alert that indicates that the patient has severe aortic stenosis when the aortic stenosis score exceeds a threshold score, or generate a second sensory alarm signal configured to generate a second sensory alert that indicates that the patient does not have severe aortic stenosis when the aortic stenosis score is below the threshold score; transmit the first sensory alarm signal or the second sensory alarm signal to the user interface; and output the first sensory alert or the second sensory alert through the user interface.

In another example, a hemodynamic monitor for detecting aortic stenosis includes an arterial blood pressure sensor. The arterial pressure sensor includes a housing, a fluid input port connected via tubing to a fluid source, a catheter-side fluid port connected to a catheter inserted within an arterial system of a patient, a pressure transducer in communication with the fluid source through the fluid port, and an I/O cable in electrical communication with the pressure transducer. The hemodynamic monitor further includes an integrated hardware unit with a system processor, a system memory, a display comprising a user interface, and an analog-to-digital (ADC) converter. The system memory includes instructions that, when executed by the system processor, are configured to: receive an electrical signal from the pressure transducer over a period of time, the electrical signal based on a pressure of the arterial system of the patient transmitted through the fluid source; convert the electrical signal to a digital signal; generate an arterial pressure waveform data of the patient based on the digital signal; extract a plurality of signal measures from the arterial pressure waveform data; extract input features from the plurality of signal measures that are indicative of an aortic stenosis score of the patient; determine the aortic stenosis score of the patient based on the extracted input features; generate a first sensory alarm signal configured to generate a first sensory alert that indicates that the patient has severe aortic stenosis when the aortic stenosis score exceeds a threshold score, or generate a second sensory alarm signal configured to generate a second sensory alert that indicates that the patient does not have severe aortic stenosis when the aortic stenosis score is below the threshold score; transmit the first sensory alarm signal or the second sensory alarm signal to the user interface; and output the first sensory alert or the second sensory alert through the user interface.

In a further example, a method for triaging a patient for aortic stenosis is disclosed. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. Input features are extracted by the hemodynamic monitor from the plurality of signal measures that are indicative of an aortic stenosis score of the patient. The hemodynamic monitor determines the aortic stenosis score of the patient based on the input features. The aortic stenosis score of the patient is outputted to a display by the hemodynamic monitor.

### BRIEF DESCRIPTION OF THE DRAWINGS

FIG. 1 is a perspective view of an example hemodynamic monitor that analyzes an arterial pressure of a patient and provides an aortic stenosis score of a patient to medical personnel.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that determines an aortic stenosis score of a patient based on a set of input features derived from signal measures of an arterial pressure waveform of the patient.

FIG. 5 is a schematic diagram of a method for triaging a patient based on an aortic stenosis score.

FIG. 6 is a diagram of a first clinical dataset, a second clinical dataset, a third clinical dataset, and a fourth clinical dataset used for data mining and machine training of the hemodynamic monitoring system.

FIG. 7 is a flow diagram for extracting a set of input features derived from signal measures of an arterial pressure waveform of a patient for training a machine learning model of a hemodynamic monitoring system.

FIG. 8 is a graph illustrating an example trace of an arterial pressure waveform including example indicia corresponding to signal measures used to extract the input features that determine the aortic stenosis score of the patient.

### DETAILED DESCRIPTION

As described herein, a hemodynamic monitoring system uses an arterial waveform of a patient to detect aortic stenosis in a patient. The hemodynamic monitoring system uses machine learning to extract sets of input features from the arterial pressure of the patient. The sets of input features are used by the hemodynamic monitoring system to determine if the patient has aortic stenosis while visiting an office of a primary care physician, while in an emergency care setting, or any other patient care environment. The hemodynamic monitoring system can even be made available over-the-counter for use at home by the patient.

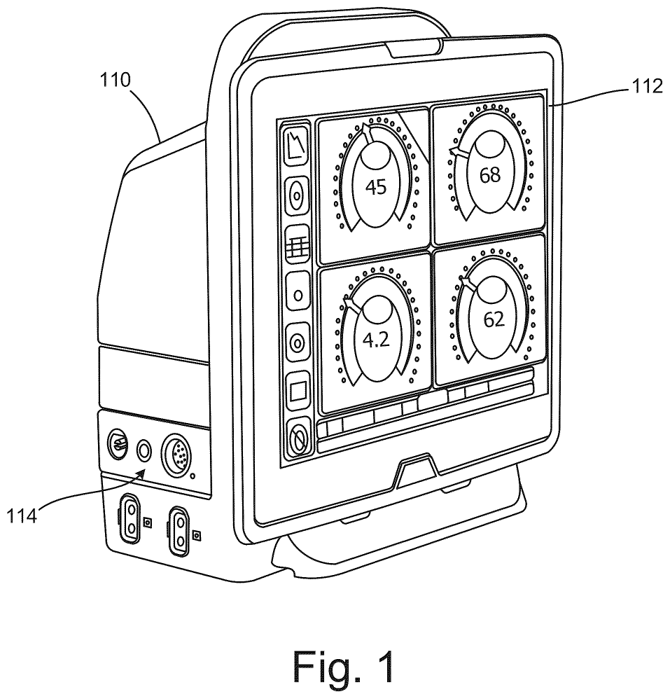

Depending on the level of aortic stenosis detected by the hemodynamic monitoring system, the hemodynamic monitoring system can raise a signal or an alarm to medical workers and/or the patient to alert the medical workers and/or the patient that the patient has mild, moderate, or severe aortic stenosis. The hemodynamic monitoring system is described in detail below with reference to FIGS. 1-8.

FIG. 1 is a perspective view of hemodynamic monitor 110 that can detect aortic stenosis in a patient. While hemodynamic monitor 110 is discussed below as detecting aortic stenosis in a patient, in other embodiments, hemodynamic monitor 110 can be used to detect additional valvular heart disorders. For example, hemodynamic monitor 110 can be used to detect aortic stenosis, mitral stenosis, mitral regurgitation, mitral valve prolapse, aortic regurgitation, and hypertrophic cardiomyopathy. As illustrated in FIG. 1, hemodynamic monitor 110 includes display 112 that, in the example of FIG. 1, presents a graphical user interface including control elements (e.g., graphical control elements) that enable user interaction with hemodynamic monitor 110. Hemodynamic monitor 110 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 110 can include I/O connectors 114. While the example of FIG. 1 illustrates five separate I/O connectors 114, it should be understood that in other examples, hemodynamic monitor 110 can include fewer than five I/O connectors or greater than five I/O connectors. In yet other examples, hemodynamic monitor 110 may not include I/O connectors 114, but rather may communicate wirelessly with various peripheral devices.

As further described below, hemodynamic monitor 110 includes one or more processors and computer-readable memory that stores aortic stenosis software code which is executable to determine an aortic stenosis score of the patient based on sensed hemodynamic data of the patient. Hemodynamic monitor 110 can receive the sensed hemodynamic data representative of an arterial pressure waveform of the patient, such as via one or more hemodynamic sensors connected to hemodynamic monitor 110 via I/O connectors 114. Hemodynamic monitor 110 executes the aortic stenosis software code to obtain, using the sensed hemodynamic data, multiple aortic stenosis profiling parameters (e.g., input features), which can include one or more vital sign parameters characterizing vital sign data of the patient, as well as differential and combinatorial parameters derived from the one or more vital sign parameters, as is further described below.

As illustrated in FIG. 1, hemodynamic monitor 10 can present a graphical user interface at display 112. Display 112 can be a liquid crystal display (LCD), a light-emitting diode (LED) display, an organic light-emitting diode (OLED) display, or other display device suitable for providing information to users in graphical form. In some examples, such as the example of FIG. 1, display 112 can be a touch-sensitive and/or presence-sensitive display device configured to receive user input in the form of gestures, such as touch gestures, scroll gestures, zoom gestures, swipe gestures, or other gesture input.

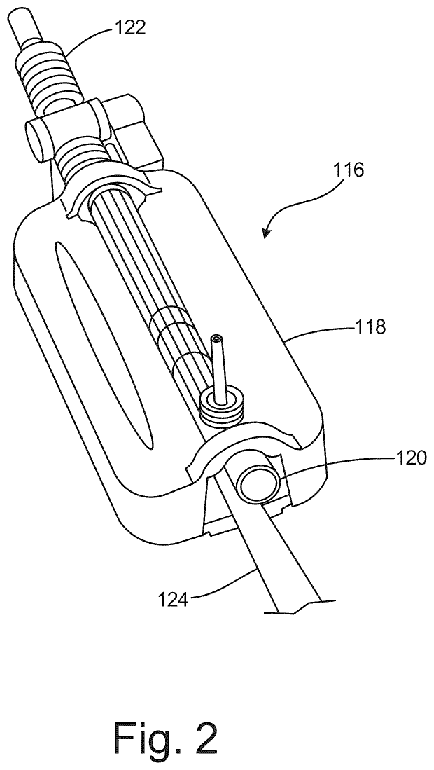

FIG. 2 is a perspective view of hemodynamic sensor 116 that can be attached to the patient for sensing hemodynamic data representative of arterial pressure of the patient. Hemodynamic sensor 116, illustrated in FIG. 2, is one example of a minimally invasive hemodynamic sensor that can be attached to the patient via, e.g., a radial arterial catheter inserted into an arm of the patient. In other examples, hemodynamic sensor 116 can be attached to the patient via a femoral arterial catheter inserted into a leg of the patient.

As illustrated in FIG. 2, hemodynamic sensor 116 includes housing 118, fluid input port 120, catheter-side fluid port 122, and I/O cable 124. Fluid input port 120 is configured to be connected via tubing or other hydraulic connection to a fluid source, such as a saline bag or other fluid input source. Catheter-side fluid port 122 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 124 is configured to connect to hemodynamic monitor 110 via, e.g., one or more of I/O connectors 114 (FIG. 1). Housing 118 of hemodynamic sensor 116 encloses one or more pressure transducers, communication circuitry, processing circuity, and corresponding electronic components to sense fluid pressure corresponding to arterial pressure of the patient that is transmitted to hemodynamic monitor 10 (FIG. 1) via I/O cable 124.

In operation, a column of fluid (e.g., saline solution) is introduced from a fluid source (e.g., a saline bag) through hemodynamic sensor 116 via fluid input port 120 to catheter-side fluid port 122 toward the catheter inserted into the patient. Arterial pressure is communicated through the fluid column to pressure sensors located within housing 116 which sense the pressure of the fluid column. Hemodynamic sensor 116 translates the sensed pressure of the fluid column to an electrical signal via the pressure transducers and outputs the corresponding electrical signal to hemodynamic monitor 10 (FIG. 1) via I/O cable 124. Hemodynamic sensor 116 therefore transmits analog sensor data (or a digital representation of the analog sensor data) to hemodynamic monitor 110 (FIG. 1) that is representative of substantially continuous beat-to-beat monitoring of the arterial pressure of the patient.

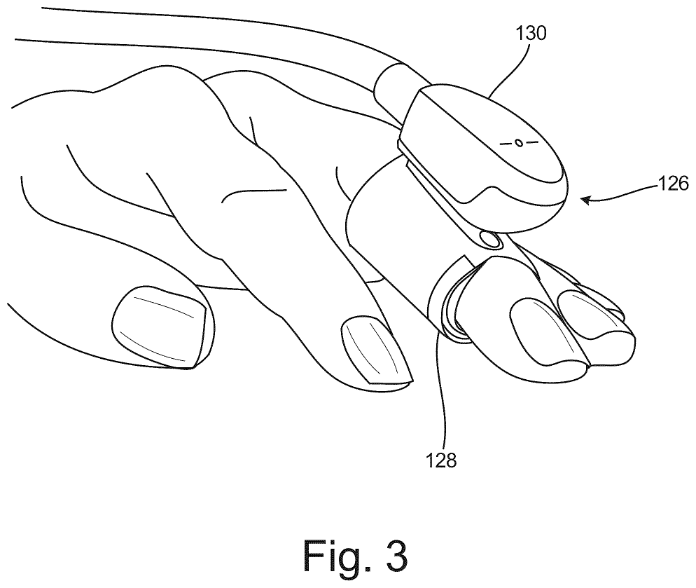

FIG. 3 is a perspective view of hemodynamic sensor 126 for sensing hemodynamic data representative of arterial pressure of the patient. Hemodynamic sensor 126, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 126 includes inflatable finger cuff 128 and heart reference sensor 130. Inflatable finger cuff 128 includes an inflatable blood pressure bladder configured to inflate and deflate as controlled by a pressure controller (not illustrated) that is pneumatically connected to inflatable finger cuff 128. Inflatable finger cuff 128 also includes an optical (e.g., infrared) transmitter and an optical receiver that are electrically connected to the pressure controller (not illustrated). The optical transmitter and the optical receiver can measure the changing volume of the arteries under the cuff in the finger. The optical transmitter and the optical receiver can be positioned to transmit and receive light therebetween through the inflatable blood pressure bladder.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 128. The pressure applied by the pressure controller to continuously maintain the unloaded volume is representative of the blood pressure in the finger and is communicated by the pressure controller to hemodynamic monitor 110 shown in FIG. 1. Heart reference sensor 130 measures the hydrostatic height difference between the level at which the finger is kept and the reference level for the pressure measurement, which typically is heart level. Accordingly, hemodynamic sensor 126 transmits sensor data that is representative of substantially continuous beat-to-beat monitoring of the arterial pressure waveform of the patient.

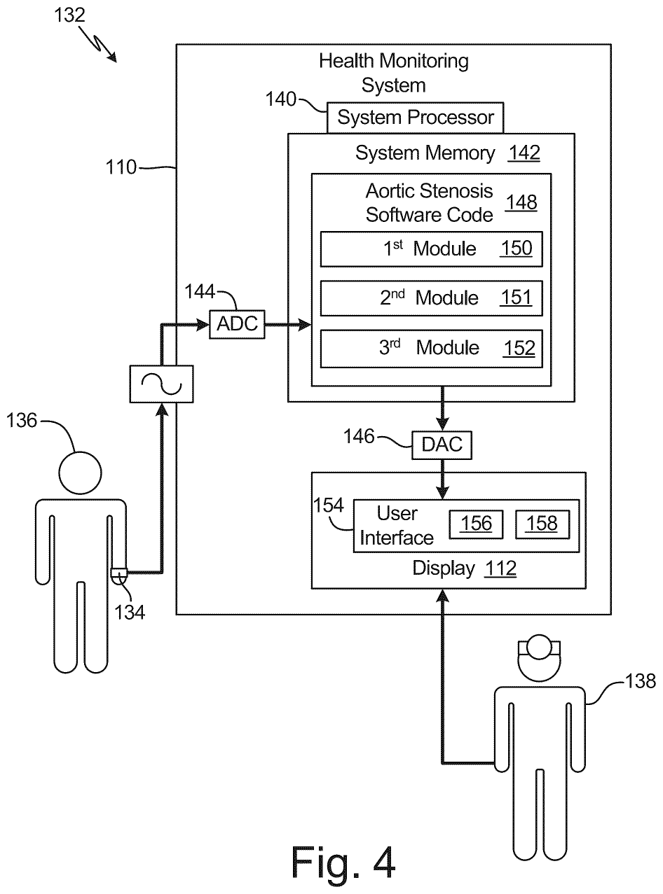

FIG. 4 is a block diagram of hemodynamic monitoring system 132 that determines an aortic stenosis score of patient 136 based on a set of aortic stenosis profiling parameters (also referred to as input features) derived from the arterial pressure of patient 136. Hemodynamic monitoring system 132 monitors the arterial pressure of patient 136 and provides the aortic stenosis score to medical worker 138. If the aortic stenosis score of patient 136 is mild, moderate, or severe, medical worker 138 can respond to the aortic stenosis score by recommending further testing or treatment to patient 136. If the aortic stenosis score of patient 136 is normal, medical worker 138 can respond to the aortic stenosis score by informing patient 136 that their aortic valve is healthy.

As illustrated in FIG. 4, hemodynamic monitoring system 132 includes hemodynamic monitor 110 and hemodynamic sensor 134. Hemodynamic monitoring system 132 can be implemented within an office of a primary care physician during a regular physical or check-up, or while in another patient care environment, such as an ICU, an OR, or any other patient care environment. Similarly, hemodynamic monitor 110 can be used at home by patient 136 for self-screening to self-determine whether patient 136 needs to see a doctor or specialist. As illustrated in FIG. 4, the patient care environment can include patient 136 and healthcare worker 138 trained to utilize hemodynamic monitoring system 132.

Hemodynamic monitor 110, as described above with respect to FIG. 1, can be, e.g., an integrated hardware unit including system processor 140, system memory 142, display 112, analog-to-digital (ADC) converter 144, and digital-to-analog (DAC) converter 146. In other examples, any one or more components and/or described functionality of hemodynamic monitor 110 can be distributed among multiple hardware units. For instance, in some examples, display 112 can be a separate display device that is remote from and operatively coupled with hemodynamic monitor 110. In general, though illustrated and described in the example of FIG. 4 as an integrated hardware unit, it should be understood that hemodynamic monitor 110 can include any combination of devices and components that are electrically, communicatively, or otherwise operatively connected to perform the functionality attributed herein to hemodynamic monitor 110.

As illustrated in FIG. 4, system memory 142 stores aortic stenosis software code 148. Aortic stenosis software code 148 includes first module 150 for extracting and calculating waveform features from the arterial pressure of patient 136, second module 151 for extracting input features from the waveform features, and third module 152 for determining the aortic stenosis score of patient 136 based on the input features. Display 112 provides user interface 154, which includes control elements 156 that enable user interaction with hemodynamic monitor 110 and/or other components of hemodynamic monitoring system 132. User interface 154, as illustrated in FIG. 4, also provides sensory alarm 158 to provide warning to medical personnel if the aortic stenosis score of patient 136 is mild, moderate, or severe. Sensory alarm 158 can be implemented as one or more of a visual alarm, an audible alarm, a haptic alarm, or other type of sensory alarm. For instance, sensory alarm 158 can be invoked as any combination of flashing and/or colored graphics shown by user interface 154 on display 112, display of the aortic stenosis score via user interface 154 on display 112, a warning sound such as a siren or repeated tone, and a haptic alarm configured to cause hemodynamic monitor 110 to vibrate or otherwise deliver a physical impulse perceptible to medical worker 138 or other user.

Hemodynamic sensor 134 can be attached to patient 136 to sense hemodynamic data representative of the arterial pressure waveform of patient 136. Hemodynamic sensor 134 is operatively connected to hemodynamic monitor 110 (e.g., electrically and/or communicatively connected via wired or wireless connection, or both) to provide the sensed hemodynamic data to hemodynamic monitor 110. In some examples, hemodynamic sensor 134 provides the hemodynamic data representative of the arterial pressure waveform of patient 136 to hemodynamic monitor 110 as an analog signal, which is converted by ADC 144 to digital hemodynamic data representative of the arterial pressure waveform. In other examples, hemodynamic sensor 134 can provide the sensed hemodynamic data to hemodynamic monitor 110 in digital form, in which case hemodynamic monitor 110 may not include or utilize ADC 144. In yet other examples, hemodynamic sensor 134 can provide the hemodynamic data representative of the arterial pressure waveform of patient 136 to hemodynamic monitor 110 as an analog signal, which is analyzed in its analog form by hemodynamic monitor 110.

Hemodynamic sensor 134 can be a non-invasive or minimally invasive sensor attached to patient 136. For instance, hemodynamic sensor 134 can take the form of minimally invasive hemodynamic sensor 116 (FIG. 2), non-invasive hemodynamic sensor 126 (FIG. 3), or other minimally invasive or non-invasive hemodynamic sensor. In some examples, hemodynamic sensor 134 can be attached non-invasively at an extremity of patient 136, such as a wrist, an arm, a finger, an ankle, a toe, or other extremity of patient 136. As such, hemodynamic sensor 134 can take the form of a small, lightweight, and comfortable hemodynamic sensor suitable for extended wear by patient 136 to provide substantially continuous beat-to-beat monitoring of the arterial pressure of patient 136 over an extended period of time, such as minutes or possibly hours. While hemodynamic sensor 134 can monitor the arterial pressure of patient 136 over an extended period of time, hemodynamic sensor 134 will only need to monitor the arterial pressure of patient 136 for a few minutes (such as 5 minutes) to provide enough data to hemodynamic monitor 110 to determine the aortic stenosis score of patient 136.

In certain examples, hemodynamic sensor 134 can be configured to sense an arterial pressure of patient 136 in a minimally invasive manner. For instance, hemodynamic sensor 134 can be attached to patient 136 via a radial arterial catheter inserted into an arm of patient 136. In other examples, hemodynamic sensor 134 can be attached to patient 136 via a femoral arterial catheter inserted into a leg of patient 136. Such minimally invasive techniques can similarly enable hemodynamic sensor 134 to provide substantially continuous beat-to-beat monitoring of the arterial pressure of patient 136 over an extended period of time, such as minutes or hours. While hemodynamic sensor 134 can monitor the arterial pressure of patient 136 over an extended period of time, hemodynamic sensor 134 will only need to monitor the arterial pressure of patient 136 for a few minutes (such as 5 minutes) to provide enough data to hemodynamic monitor 110 to determine the aortic stenosis score of patient 136.

System processor 140 is a hardware processor configured to execute aortic stenosis software code 148, which implements first module 150, second module 151, and third module 152 to generate the aortic stenosis score for patient 136. Examples of system processor 140 can include any one or more of a microprocessor, a controller, a digital signal processor (DSP), an application specific integrated circuit (ASIC), a field-programmable gate array (FPGA), or other equivalent discrete or integrated logic circuitry.

System memory 142 can be configured to store information within hemodynamic monitor 110 during operation. System memory 142, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 142 can include volatile and non-volatile computer-readable memories. Examples of volatile memories can include random access memories (RAM), dynamic random access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 112 can be a liquid crystal display (LCD), a light-emitting diode (LED) display, an organic light-emitting diode (OLED) display, or other display device suitable for providing information to users in graphical form. User interface 154 can include graphical and/or physical control elements that enable user input to interact with hemodynamic monitor 110 and/or other components of hemodynamic monitoring system 132. In some examples, user interface 154 can take the form of a graphical user interface (GUI) that presents graphical control elements presented at, e.g., a touch-sensitive and/or presence sensitive display screen of display 112. In such examples, user input can be received in the form of gesture input, such as touch gestures, scroll gestures, zoom gestures, or other gesture input. In certain examples, user interface 154 can take the form of and/or include physical control elements, such as a physical buttons, keys, knobs, or other physical control elements configured to receive user input to interact with components of hemodynamic monitoring system 132.

In operation, hemodynamic sensor 134 senses hemodynamic data representative of an arterial pressure waveform of patient 136. Hemodynamic sensor 134 provides the hemodynamic data (e.g., as analog sensor data), to hemodynamic monitor 110. ADC 144 converts the analog hemodynamic data to digital hemodynamic data representative of the arterial pressure waveform of the patient.

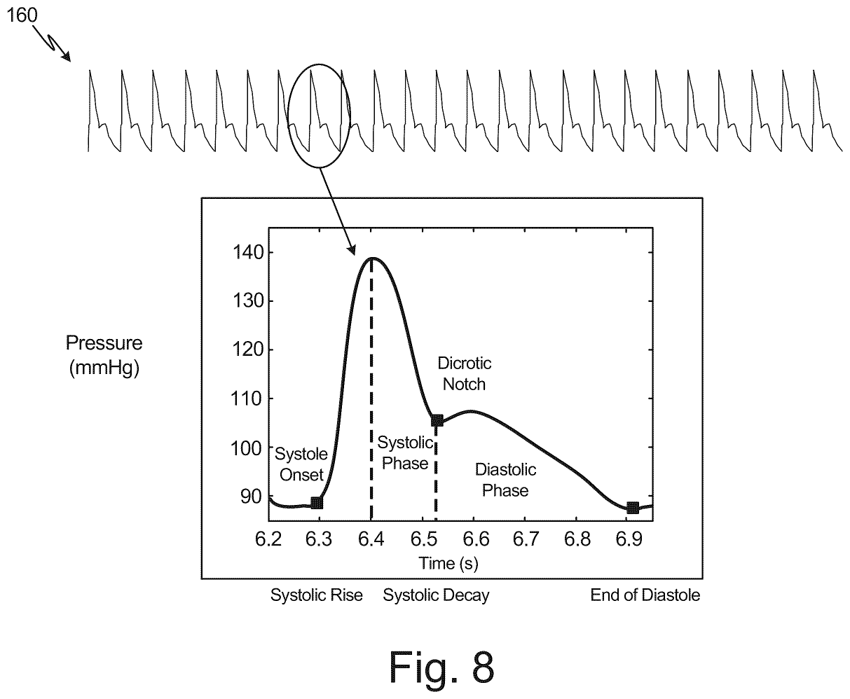

System processor 140 executes aortic stenosis software code 148 to determine, using the received hemodynamic data, the aortic stenosis score for patient 136. For instance, system processor 140 can execute first module 150 to perform waveform analysis of the hemodynamic data to determine a plurality of signal measures. The plurality of signal measures are waveform features and hemodynamic effects that characterize individual cardiac cycles of the arterial pressure waveform of the patient. The plurality of signal measures is discussed in greater detail below in the discussion of FIG. 8. System processor 140 executes second module 151 to extract input features from the plurality of signal measures that are determinative of the aortic stenosis score of patient 136. System processor 140 executes third module 152 to determine, based on the input features, the aortic stenosis score of patient 136.

If the aortic stenosis score of patient 136 is within a first range (such as zero to forty), system processor 140 invokes sensory alarm 158 of user interface 154 to send a first sensory signal to alert medical worker 138 that patient 136 has a healthy aortic valve. Additional screening or examination of patient 136 for aortic stenosis is unlikely when hemodynamic monitor 110 determines patient 136 has a normal aortic stenosis score.

If system processor 140 and third module 152 determine that the aortic stenosis score of patient 136 is within a second range (such as forty-one to sixty), system processor 140 invokes sensory alarm 158 of user interface 154 to send a second sensory signal to alert medical worker 138 that patient 136 has a mild aortic stenosis score indicative of an aortic valve area greater than 1.5 cm². Medical worker 138 can respond to the mild aortic stenosis score of patient 136 by recommending patient 136 undergo further tests and examinations to verify the health of the aortic valve of patient 136.

If system processor 140 and third module 152 determine that the aortic stenosis score of patient 136 is within a third range (such as sixty-one to eighty), system processor 140 invokes sensory alarm 158 of user interface 154 to send a third sensory signal to alert medical worker 138 that patient 136 has a moderate aortic stenosis score indicative of an aortic valve area within 1.0 cm²-1.5 cm². Medical worker 138 can respond to the moderate aortic stenosis score of patient 136 by recommending patient 136 quickly undergo further tests and examinations to verify the health of the aortic valve of patient 136.

If system processor 140 and third module 152 determine that the aortic stenosis score of patient 136 is within a fourth range (such as eighty-one to one-hundred), system processor 140 invokes sensory alarm 158 of user interface 154 to send a fourth sensory signal to alert medical worker 138 that patient 136 has a severe aortic stenosis score indicative of an aortic valve area less than 1.0 cm². Medical worker 138 can respond to the moderate aortic stenosis score of patient 136 by recommending patient 136 immediately undergo further tests and examinations to verify the health of the aortic valve of patient 136 and to seek treatment and/or repair of the aortic valve. In this manner, hemodynamic monitor 110 functions as a screening tool that can be used in the office of a primary care physician for detecting and discovering mild, moderate, or severe aortic stenosis in patient 136 during routine physical examinations. Similarly, hemodynamic monitor 110 can be used at home by patient 136 for self-screening to self-determine whether patient 136 needs to see a doctor or specialist.

In some embodiments, system processor 140 can determine multiple subsets of the input features, with each subset of the input features being related to a different level or range of aortic stenosis score. For example, system processor 140 can execute first module 150 to perform waveform analysis of the hemodynamic data to determine a plurality of signal measures. System processor 140 executes second module 151 to extract a first subset, a second subset, a third subset, and a fourth subset of the input features from the plurality of signal measures of patient 136. The first subset of the input features are those input features that third module 152 will use to determine whether patient 136 has a normal aortic stenosis score. The second subset of the input features are those input features that third module 152 will use to determine whether patient 136 has a mild aortic stenosis score. The third subset of the input features are those input features that third module 152 will use to determine whether patient 136 has a moderate aortic stenosis score. The fourth subset of the input features are those input features that third module 152 will use to determine whether patient 136 has a severe aortic stenosis score. System processor 140 can execute first module 150 to extract a single batch of the plurality of signal measures for a given unit of time, and that single batch of signal measures can be used by second module 151 to extract all of the first subset, the second subset, the third subset, and the fourth subset of the input features for that unit of time. Second module 151 can extract all of the first subset, the second subset, the third subset, and the fourth subset of the input features concurrently from the plurality of signal measures. System processor 140 can execute third module 152 to concurrently calculate probabilities of a normal aortic stenosis score, a mild aortic stenosis score, a moderate aortic stenosis score, and a severe aortic stenosis score for patient 136.

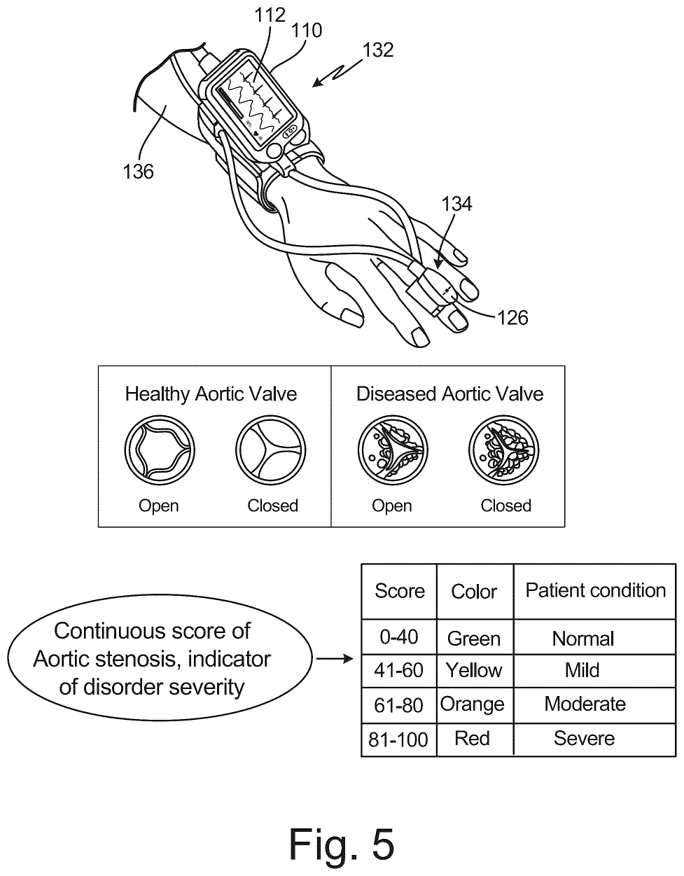

Aortic stenosis software code 148 of hemodynamic monitor 110 can utilize, in some examples, a multi-classification-type machine learning model with four labels: normal aortic function verses mild aortic stenosis versus moderate aortic stenosis versus severe aortic stenosis. For example, processor 140 can output to display 112 the normal aortic stenosis score of patient 136 along with the mild, moderate, and severe aortic stenosis scores of patient 136, so that all four probabilities are compared together: the probability patient 136 has a normal aortic function, the probability that patient 136 has mild aortic stenosis, the probability that patient 136 has moderate aortic stenosis, and the probability that patient 136 has severe aortic stenosis. With the normal aortic stenosis score, the mild aortic stenosis score, the moderate aortic stenosis score, and the severe aortic stenosis score of patient 136 together on display 112 of hemodynamic monitor 110, medical worker 138 can better understand and cross-check whether patient 136 has a normal aortic function versus mild aortic stenosis, moderate aortic stenosis, or severe aortic stenosis. As discussed below with reference to FIG. 5, hemodynamic monitor 110 is a fast and efficient tool for screening and triaging patients 36 before referring patients 136 for more lengthy and costly examination.

FIG. 5 shows a perspective view of hemodynamic monitoring system 132 and a schematic diagram of a method for triaging patient 136 based on the aortic stenosis score of patient 136. As shown in FIG. 5, hemodynamic monitoring system 132 includes hemodynamic monitor 110 and hemodynamic sensor 134. In the embodiment of FIG. 5, hemodynamic sensor 134 is non-invasive hemodynamic sensor 126 (described in detail above with reference to FIG. 3) that can be attached to patient 136 via one or more finger cuffs to sense data representative of arterial pressure of patient 136. In the embodiment of FIG. 5, hemodynamic monitor 110 is a compact wearable unit that can be strapped to an arm of patient 136 and connected to hemodynamic sensor 134 to receive the sensed data representative of arterial pressure of patient 136. The embodiment of hemodynamic monitoring system 132 of FIG. 5 can operate and function as described above with reference to FIG. 4 to determine the aortic stenosis score of patient 136. During a routine physical at an office of a primary care physician, patient 136 can be quickly tested and triaged for aortic stenosis by connecting hemodynamic monitoring system 132 to a hand and arm of patient 136 and feeding the sensed hemodynamic data of patient 136 into hemodynamic monitor 110. After a few minutes (such as five minutes) of feeding the sensed hemodynamic data of patient 136 into hemodynamic monitor 110, hemodynamic monitor 110 will output to display 112 the aortic stenosis score of patient 136. In some embodiments, hemodynamic monitor 110 can also output the aortic stenosis score of patient 136 to a mobile device of patient 136.

Hemodynamic monitor 110 can color-code and/or score the aortic stenosis score of patient 136 in display 112 depending on whether the aortic stenosis score is normal, mild, moderate, or severe. As noted above with reference to FIG. 4, a normal aortic stenosis score can be within zero and forty, a mild aortic stenosis score can within forty and sixty, a moderate aortic stenosis score can be within sixty-one and eighty, and a severe aortic stenosis score can be within eighty-one and one-hundred. If hemodynamic monitor 110 determines that patient 136 has a normal aortic stenosis score, hemodynamic monitor 110 can output a green-colored score with a numerical value within zero to forty to display 112. If hemodynamic monitor 110 determines that patient 136 has a mild aortic stenosis score, hemodynamic monitor can output a yellow-colored score with a numerical value within forty-one to sixty to display 112. If hemodynamic monitor 110 determines that patient 136 has a moderate aortic stenosis score, hemodynamic monitor can output an orange-colored score with a numerical value within sixty-one to eighty to display 112. If hemodynamic monitor 110 determines that patient 136 has a severe aortic stenosis score, hemodynamic monitor 110 can output a red-colored score with a numerical value within eighty-one to one-hundred to display 112. The above listed aortic stenosis scores are provided by way of example and can be modified or adjusted in other embodiments while remaining within the scope of this disclosure.

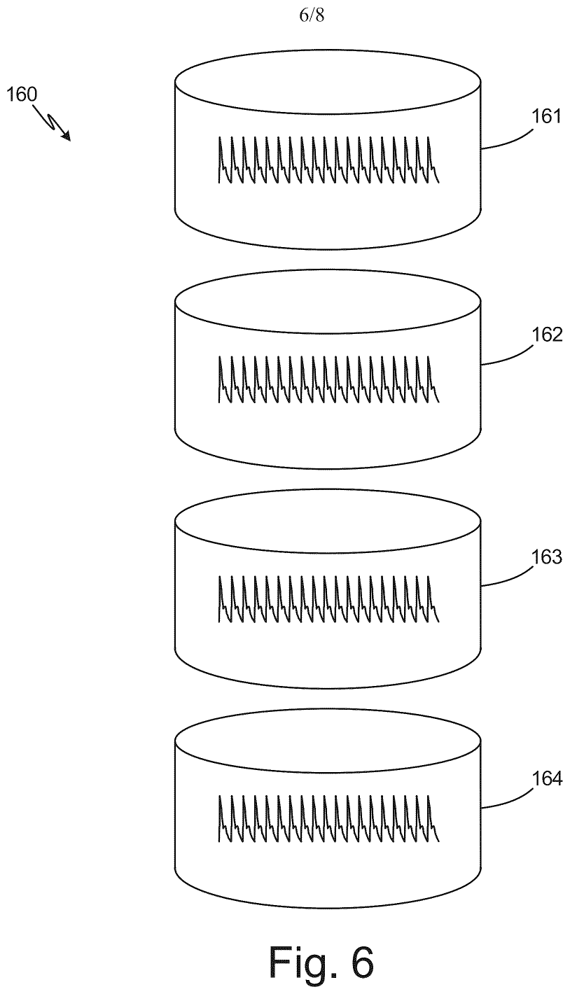

Medical worker 138 (shown in FIG. 4) in this scenario can be a primary care physician or nurse that is performing the routine physical examination of patient 136. Once hemodynamic monitor 110 outputs the aortic stenosis score of patient 136 to display 112 after monitoring and processing the sensed hemodynamic data of patient 136 for several minutes, medical worker 138 can triage patient 136 based on the aortic stenosis score of patient 136. If the aortic stenosis score of patient 136 is indicated as being severe on display 112, medical worker 138 can inform patient 136 that patient 136 should seek further testing, examination, and treatment immediately from a cardiovascular specialist. Medical worker 138 can then refer patient 136 to a cardiovascular specialist to undergo more intensive examination, such as an echocardiogram or electrocardiogram, on an expedited schedule. If the aortic stenosis score of patient 136 in display 112 is mild or moderate, medical worker 138 can inform patient 136 that patient 136 should seek further testing, examination, and treatment soon from a cardiovascular specialist, and can refer patient 136 to a specialist on a less expedited schedule than a patient with a severe aortic stenosis score. If the aortic stenosis score of patient 136 in display 112 is normal, medical worker 138 can inform patient 136 that patient 136 has a healthy aortic valve and that no additional testing is needed. As discussed below with reference to FIGS. 6-8, a machine learning model of hemodynamic monitor 110 can be trained using clinical data sets to recognize the input features in the arterial pressure waveform of patient 136 and use those input features to determine the aortic stenosis score of patient 136.

FIG. 6 is a diagram of clinical data 160 used for data mining and machine training of hemodynamic monitor 110 of hemodynamic monitoring system 132. Clinical data 160 includes first clinical dataset 161, second clinical dataset 162, third clinical dataset 163, and fourth clinical dataset 164.

First clinical dataset 161 contains a collection of arterial pressure waveforms recorded from a first group of individuals who each have been confirmed as having normal aortic valve function. First clinical dataset 161 can be collected from the first group of individuals by invasive hemodynamic sensors, such as hemodynamic sensor 116 shown in FIG. 2, or collected by non-invasive hemodynamic sensors, such as hemodynamic sensor 126 shown in FIG. 3. When each individual in the first clinical dataset 161 is connected to a hemodynamic sensor, the hemodynamic sensor records an arterial pressure waveform of that individual and the arterial pressure waveform of that individual is tagged with a first label so that arterial pressure waveform can eventually be added to first clinical dataset 161. Adding the first label to the arterial pressure waveforms of the individuals in the first group also allows first clinical dataset 161 to be collected and stored in a common location with second clinical dataset 162, third clinical dataset 163, and fourth clinical dataset 164 without the arterial pressure waveforms of first clinical dataset 161 being lost or confused amongst the arterial pressure waveforms of second clinical dataset 162, third clinical dataset 163, and fourth clinical dataset 164.

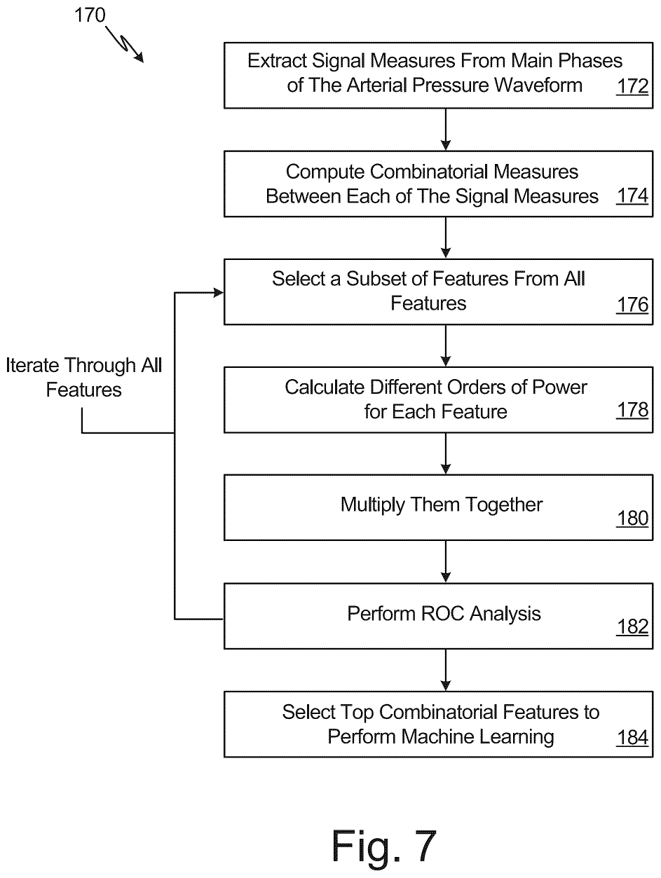

After the arterial pressure waveforms of first clinical dataset 161 have been collected and labeled with first label, the arterial pressure waveforms of first clinical dataset 161 are ready for use for data mining and machine training of hemodynamic monitor 110. The arterial pressure waveforms of first clinical dataset 161 are data mined and used to machine train hemodynamic monitor 110 to determine the first subset of the input features. As discussed above with reference to FIG. 4, the first subset of the input features are those input features that third module 152 will use to determine whether patient 136 has a normal aortic stenosis score. As will be discussed further below with reference to FIGS. 7-8, waveform analysis is performed on first clinical dataset 61 to calculate a plurality of signal measures which are then used to compute the first subset of the input features that best detect and measure normal aortic valve function from an arterial pressure waveform.

Second clinical dataset 162 contains a collection of arterial pressure waveforms recorded from a second group of individuals who each have a confirmed case of mild aortic stenosis, wherein mild aortic stenosis is defined as an aortic valve area greater than 1.5 cm². Second clinical dataset 162 can be collected from the second group of individuals by invasive hemodynamic sensors, such as hemodynamic sensor 116 shown in FIG. 2, or collected by non-invasive hemodynamic sensors, such as hemodynamic sensor 126 shown in FIG. 3. When each individual in the second clinical dataset 162 is connected to a hemodynamic sensor, the hemodynamic sensor records an arterial waveform of that individual and the arterial waveform of that individual is tagged with a second label so that arterial waveform can eventually be added to second clinical dataset 162. Adding the second label to the arterial pressure waveforms of the individuals in the second group also allows second clinical dataset 162 to be collected and stored in a common location with first clinical dataset 161, third clinical dataset 163, and fourth clinical dataset 164 without the arterial pressure waveforms of second clinical dataset 162 being lost or confused amongst the arterial pressure waveforms of first clinical dataset 161, third clinical dataset 163, and fourth clinical dataset 164.

After the arterial pressure waveforms of second clinical dataset 162 have been collected and labeled with second label, the arterial pressure waveforms of second clinical dataset 162 are ready for use for data mining and machine training of hemodynamic monitor 110. The arterial pressure waveforms of second clinical dataset 162 are data mined and used to machine train hemodynamic monitor 110 to determine the second subset of the input features. As discussed above with reference to FIG. 4, the second subset of the input features are those input features that third module 152 will use to determine whether patient 136 has a mild aortic stenosis score. As will be discussed further below with reference to FIGS. 7-8, waveform analysis is performed on second clinical dataset 162 to calculate a plurality of signal measures which are then used to compute the second subset of the input features that best detect and measure mild aortic stenosis (aortic valve area greater than 1.5 cm²) from an arterial pressure waveform.

Third clinical dataset 163 contains a collection of arterial pressure waveforms recorded from a third group of individuals who each have a confirmed case of moderate aortic stenosis, with moderate aortic stenosis being defined as an aortic valve area between 1.0 cm² and 1.5 cm². Third clinical dataset 163 can be collected from the third group of individuals by invasive hemodynamic sensors, such as hemodynamic sensor 116 shown in FIG. 2, or collected by non-invasive hemodynamic sensors, such as hemodynamic sensor 126 shown in FIG. 3. When each individual in the third clinical dataset 163 is connected to a hemodynamic sensor, the hemodynamic sensor records an arterial pressure waveform of that individual and the arterial pressure waveform of that individual is tagged with a third label so that arterial pressure waveform can eventually be added to third clinical dataset 163. Adding the third label to the arterial pressure waveforms of the individuals in the third group also allows third clinical dataset 163 to be collected and stored in a common location with first clinical dataset 161, second clinical dataset 162, and fourth clinical dataset 164 without the arterial pressure waveforms of third clinical dataset 163 being lost or confused amongst the arterial pressure waveforms of first clinical dataset 161, second clinical dataset 162, and fourth clinical dataset 164.

After the arterial pressure waveforms of third clinical dataset 163 have been collected and labeled with third label, the arterial pressure waveforms of third clinical dataset 162 are ready for use for data mining and machine training of hemodynamic monitor 110. The arterial pressure waveforms of third clinical dataset 163 are data mined and used to machine train hemodynamic monitor 110 to determine the third subset of the input features. As discussed above with reference to FIG. 4, the third subset of the input features are those input features that third module 152 will use to determine whether patient 136 has a moderate aortic stenosis score. As will be discussed further below with reference to FIGS. 7-8, waveform analysis is performed on third clinical dataset 162 to calculate a plurality of signal measures which are then used to compute the third subset of the input features that best detect and measure moderate aortic stenosis (aortic valve area within 1.0 cm² and 1.5 cm²) from an arterial pressure waveform.

Fourth clinical dataset 164 contains a collection of arterial pressure waveforms recorded from a fourth group of individuals who each have a confirmed case of severe aortic stenosis, with severe aortic stenosis being defined as an aortic valve area less than 1.0 cm². Fourth clinical dataset 164 can be collected from the fourth group of individuals by invasive hemodynamic sensors, such as hemodynamic sensor 116 shown in FIG. 2, or collected by non-invasive hemodynamic sensors, such as hemodynamic sensor 126 shown in FIG. 3. When each individual in the fourth clinical dataset 164 is connected to a hemodynamic sensor, the hemodynamic sensor records an arterial pressure waveform of that individual and the arterial pressure waveform of that individual is tagged with a fourth label so that arterial pressure waveform can eventually be added to fourth clinical dataset 164. Adding the fourth label to the arterial pressure waveforms of the individuals in the fourth group also allows fourth clinical dataset 164 to be collected and stored in a common location with first clinical dataset 161, second clinical dataset 162, and third clinical dataset 163 without the arterial pressure waveforms of fourth clinical dataset 164 being lost or confused amongst the arterial pressure waveforms of first clinical dataset 161, second clinical dataset 162, and third clinical dataset 163.

After the arterial pressure waveforms of fourth clinical dataset 164 have been collected and labeled with fourth label, the arterial pressure waveforms of fourth clinical dataset 164 are ready for use for data mining and machine training of hemodynamic monitor 110. The arterial pressure waveforms of fourth clinical dataset 164 are data mined and used to machine train hemodynamic monitor 110 to determine the fourth subset of the input features. As discussed above with reference to FIG. 4, the fourth subset of the input features are those input features that third module 152 will use to determine whether patient 136 has a severe aortic stenosis score. As will be discussed further below with reference to FIGS. 7-8, waveform analysis is performed on fourth clinical dataset 164 to calculate a plurality of signal measures which are then used to compute the fourth subset of the input features that best detect and measure severe aortic stenosis (aortic valve area less than 1.0 cm²) from an arterial pressure waveform.

FIG. 7 is a flow diagram of method 170 for data mining clinical data 160 from FIG. 6 for machine training the machine learning model of hemodynamic monitor 110. Method 170 in FIG. 7 will be discussed while also referencing FIG. 8. Method 170 is applied to each of first clinical dataset 161, second clinical dataset 162, third clinical dataset 163, and fourth dataset 164 to train hemodynamic monitor 110 to find the input features (including the first subset, the second subset, the third subset, and the fourth subset of the input features) previously described with reference to FIGS. 4 and 6. Method 170 will be described as applied to the arterial pressure waveforms of first clinical dataset 161.

To machine train hemodynamic monitor 110 to identify the first subset of the input features described in FIG. 4, the first subset of input features are first determined by applying method 170 to the arterial pressure waveforms of first clinical dataset 161 of clinical data 160. First step 172 of method 170 is to perform waveform analysis of the arterial pressure waveforms collected in first clinical dataset 161 to calculate a plurality of signal measures of first clinical dataset 161. Performing waveform analysis of the arterial pressure waveforms of first clinical dataset 161 can include identifying individual cardiac cycles in each of the arterial pressure waveforms of first clinical dataset 161. FIG. 8 provides an example graph illustrating an example trace of an arterial pressure waveform with an individual cardiac cycle identified and enlarged. Next, performing waveform analysis of the arterial pressure waveforms of first clinical dataset 161 can include identifying a dicrotic notch in each of the individual cardiac cycles of each of the arterial pressure waveforms of first clinical dataset 161, similar to the example shown in FIG. 8. Next, the waveform analysis on the arterial pressure waveforms of first clinical dataset 161 includes identifying a systolic rise phase, a systolic decay phase, and a diastolic phase in each of the individual cardiac cycles of each of the arterial pressure waveforms of first clinical dataset 161, similar to the example shown in FIG. 8.

Signal measures are extracted from each of the systolic rise phase, the systolic decay phase, and the diastolic phase from each of the individual cardiac cycles of each of the arterial pressure waveforms of first clinical dataset 161. The signal measures can correspond to hemodynamic effects from each of the systolic rise phase, the systolic decay phase, and the diastolic phase from each of the individual cardiac cycles. Those hemodynamic effects can include contractility, aortic compliance, stroke volume, vascular tone, afterload, and full cardiac cycle. The signal measures calculated or extracted by the waveform analysis of first step 172 of method 170 includes a mean, a maximum, a minimum, a duration, an area, a standard deviation, derivatives, and/or morphological measures from each of the systolic rise phase, the systolic decay phase, and the diastolic phase from each of the individual cardiac cycles. The signal measures can also include heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles of each of the arterial pressure waveforms of first clinical dataset 161.

After the signal measures are determined for first clinical dataset 161, step 174 of method 170 is performed on the signal measures of first clinical dataset 161. Step 174 of method 170 computes combinatorial measures between the signal measures of first clinical dataset 161. Computing the combinatorial measures between the signal measures of first clinical dataset 161 can include performing steps 176, 178, 180, and 182 shown in FIG. 7 on all the signal measures of first clinical dataset 161. Step 176 is performed by arbitrarily selecting a subset of signal measures (such as subset of signal measures) from the signal measures of first clinical dataset 161. Next, different orders of power are calculated for each signal measure of the subset of signal measures to generate powers of the subset of signal measures, as shown in step 178 of FIG. 7. In step 180 of FIG. 7, the powers of the subset of signal measures are then multiplied together to generate the product of the powers of the subset of signal measures. Step 182 includes performing receiver operating characteristic (ROC) analysis of the product to arrive at a combinatorial measure for the subset of signal measures. Steps 176, 178, 180, and 182 are repeated until all of the combinatorial measures have been computed between all of the signal measures of first clinical dataset 161. The final step 184 includes selecting the signal measures with most predictive top combinatorial measures (i.e., combinatorial measures satisfying a threshold prediction criteria) as top signal measures for first clinical dataset 161 and are labeled as the first subset of the input features. With the first subset of the input features determined, hemodynamic monitor 110 is trained or programmed to perform waveform analysis on the arterial pressure waveform of patient 136 (shown in FIG. 4) and extract the first subset of the input features from the arterial pressure waveform of patient 136, and use the first subset of the input features to determine whether patient 136 has a normal aortic stenosis score.

Similar to how method 170 was applied to the arterial pressure waveforms of first clinical dataset 161 to determine the first subset of the input features, method 170 is applied to second clinical dataset 162 to determine the second subset of the input features. Method 170 is also applied to third clinical dataset 163 to determine the third subset of the input features. Method 170 is also applied to fourth clinical dataset 164 to determine the fourth subset of the input features.

### Discussion of Possible Embodiments

The following are non-exclusive descriptions of possible embodiments of the present invention.

In one example, a method is disclosed for triaging a patient for aortic stenosis. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The method further includes extracting, by the hemodynamic monitor, input features from the plurality of signal measures that are indicative of an aortic stenosis score of the patient. The hemodynamic monitor determines the aortic stenosis score of the patient based on the input features and outputs the aortic stenosis score of the patient to a display and/or mobile device.

The method of the preceding paragraph can optionally include, additionally and/or alternatively, any one or more of the following features, configurations, and/or additional components listed below.

A further embodiment of the foregoing method, further comprising: alerting the patient and/or medical personnel that the aortic stenosis score is normal when the aortic stenosis score is within a first range; alerting the patient and/or the medical personnel that the aortic stenosis score is mild when the aortic stenosis score is within a second range; alerting the patient and/or the medical personnel that the aortic stenosis score is moderate when the aortic stenosis score is within a third range; and alerting the patient and/or the medical personnel that the aortic stenosis score is severe when the aortic stenosis score is within a fourth range.

A further embodiment of the foregoing method, further comprising: training the hemodynamic monitor for determining the aortic stenosis score of the patient, wherein training the hemodynamic monitor comprises: collecting a first clinical dataset containing arterial pressure waveforms from a first group of individuals with normal aortic valve function; collecting a second clinical dataset containing arterial pressure waveforms from a second group of individuals with mild aortic stenosis, wherein mild aortic stenosis is defined as an aortic valve area greater than 1.5 cm²; collecting a third clinical dataset containing arterial pressure waveforms from a third group of individuals with moderate aortic stenosis, wherein moderate aortic stenosis is defined as an aortic valve area between 1 cm² and 1.5 cm²; collecting a fourth clinical dataset containing arterial pressure waveforms from a fourth group of individuals with severe aortic stenosis, wherein severe aortic stenosis is defined as an aortic valve area less than 1 cm²; performing waveform analysis of the arterial pressure waveforms of the first clinical dataset, the second clinical dataset, the third clinical dataset, and the fourth clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

In another example, a system for triaging a patient for aortic stenosis includes a hemodynamic sensor that produces hemodynamic data representative of an arterial pressure waveform of the patient. The system further includes a system memory that stores aortic stenosis software code. A user interface of the system includes a display to show an aortic stenosis score of the patient to medical personnel. The system includes a processor that is configured to execute the aortic stenosis software code to: perform waveform analysis of the hemodynamic data to determine a plurality of signal measures; extract input features from the plurality of signal measures that are indicative of the aortic stenosis score of the patient; determine, based on the input features, the aortic stenosis score of the patient; and output the aortic stenosis score of the patient to the display of the user interface.

The system of the preceding paragraph can optionally include, additionally and/or alternatively, any one or more of the following features, configurations, and/or additional components listed below.

A further embodiment of the foregoing system, wherein the input features of the aortic stenosis software code are determined by machine training, wherein the machine training comprises: collecting a first clinical dataset containing arterial pressure waveforms from a first group of individuals with normal aortic valve function; collecting a second clinical dataset containing arterial pressure waveforms from a second group of individuals with mild aortic stenosis, wherein mild aortic stenosis is defined as an aortic valve area greater than 1.5 cm²; collecting a third clinical dataset containing arterial pressure waveforms from a third group of individuals with moderate aortic stenosis, wherein moderate aortic stenosis is defined as an aortic valve area between 1 cm² and 1.5 cm²; collecting a fourth clinical dataset containing arterial pressure waveforms from a fourth group of individuals with severe aortic stenosis, wherein severe aortic stenosis is defined as an aortic valve area less than 1 cm²; performing waveform analysis of the arterial pressure waveforms of the first clinical dataset, the second clinical dataset, the third clinical dataset, and the fourth clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing system, wherein performing waveform analysis of the labeled arterial pressure waveforms of the first clinical dataset, the second clinical dataset, the third clinical dataset, and the fourth clinical dataset to calculate the plurality of waveform signal measures comprises: identifying individual cardiac cycles in each of the arterial pressure waveforms of the first clinical dataset, the second clinical dataset, the third clinical dataset, and the fourth clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing system, wherein the plurality of waveform signal measures corresponds to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing system, wherein the plurality of waveform signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles.

A further embodiment of the foregoing system, wherein the plurality of waveform signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing system, wherein computing the combinatorial measures between the plurality of waveform signal measures of the first clinical dataset, the second clinical dataset, the third clinical dataset, and the fourth clinical dataset comprises: performing step one by arbitrarily selecting a subset of signal measures from the plurality of waveform signal measures; performing step two by calculating different orders of power for each signal measure of the subset of signal measures to generate powers of the subset of signal measures; performing step three by multiplying the powers of the signal measures of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing system, wherein the hemodynamic sensor is a noninvasive hemodynamic sensor that is attachable to an extremity of the patient.

A further embodiment of the foregoing system, wherein the hemodynamic sensor is a minimally invasive arterial catheter based hemodynamic sensor.

A further embodiment of the foregoing system, wherein the hemodynamic sensor produces the hemodynamic data as an analog hemodynamic sensor signal representative of the arterial pressure waveform of the patient.

A further embodiment of the foregoing system, further comprising: an analog-to-digital converter that converts the analog hemodynamic sensor signal to digital hemodynamic data representative of the arterial pressure waveform of the patient.

In another example, a method is disclosed for triaging a patient for aortic stenosis. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The method further includes extracting, by the hemodynamic monitor, input features from the plurality of signal measures that are indicative of an aortic stenosis score of the patient. The hemodynamic monitor determines the aortic stenosis score of the patient based on the input features and outputs the aortic stenosis score of the patient to a display. The hemodynamic monitor alerts the patient and/or medical personnel that the aortic stenosis score is normal when the aortic stenosis score is within a first range. The hemodynamic monitor alerts the patient and/or the medical personnel that the aortic stenosis score is mild when the aortic stenosis score is within a second range. The hemodynamic monitor alerts the patient and/or the medical personnel that the aortic stenosis score is moderate when the aortic stenosis score is within a third range. The hemodynamic monitor alerts the patient and/or the medical personnel that the aortic stenosis score is severe when the aortic stenosis score is within a fourth range.

The method of the preceding paragraph can optionally include, additionally and/or alternatively, any one or more of the following features, configurations, and/or additional components listed below.

A further embodiment of the foregoing method, further comprising: training the hemodynamic monitor for determining the aortic stenosis score of the patient, wherein training the hemodynamic monitor comprises: collecting a first clinical dataset containing arterial pressure waveforms from a first group of individuals with normal aortic valve function; labeling each of the arterial pressure waveforms of the first clinical dataset with a first label; performing waveform analysis of the labeled arterial pressure waveforms of the first clinical dataset to calculate a plurality of waveform signal measures of the first clinical dataset; and determining a first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features.

A further embodiment of the foregoing method, wherein training the hemodynamic monitor for determining the aortic stenosis score of the patient further comprises: collecting a second clinical dataset containing arterial pressure waveforms from a second group of individuals with mild aortic stenosis, wherein mild aortic stenosis is defined as an aortic valve area greater than 1.5 cm²; labeling each of the arterial pressure waveforms of the second clinical dataset with a second label; performing waveform analysis of the labeled arterial pressure waveforms of the second clinical dataset to calculate a plurality of waveform signal measures of the second clinical dataset; and determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features.

A further embodiment of the foregoing method, wherein training the hemodynamic monitor for determining the aortic stenosis score of the patient further comprises: collecting a third clinical dataset containing arterial pressure waveforms from a third group of individuals with moderate aortic stenosis, wherein moderate aortic stenosis is defined as an aortic valve area between 1 cm² and 1.5 cm²; labeling each of the arterial pressure waveforms of the third clinical dataset with a third label; performing waveform analysis of the labeled arterial pressure waveforms of the third clinical dataset to calculate a plurality of waveform signal measures of the third clinical dataset; and determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features.

A further embodiment of the foregoing method, wherein training the hemodynamic monitor for determining the aortic stenosis score of the patient further comprises: collecting a fourth clinical dataset containing arterial pressure waveforms from a fourth group of individuals with severe aortic stenosis, wherein severe aortic stenosis is defined as an aortic valve area less than 1 cm²; labeling each of the arterial pressure waveforms of the fourth clinical dataset with a fourth label; performing waveform analysis of the labeled arterial pressure waveforms of the fourth clinical dataset to calculate a plurality of waveform signal measures of the fourth clinical dataset; and determining a fourth subset of the input features by computing combinatorial measures between the plurality of waveform signal measures of the fourth clinical dataset and selecting top signal measures from the plurality of waveform signal measures of the fourth clinical dataset with most predictive combinatorial measures and labeling the top signal measures of the fourth clinical data set as the fourth subset of the input features.

A further embodiment of the foregoing method, wherein performing waveform analysis of the labeled arterial pressure waveforms of the first clinical dataset to calculate the plurality of waveform signal measures of the first clinical dataset comprises: identifying individual cardiac cycles in each of the arterial pressure waveforms of the first clinical dataset; identifying a dicrotic notch in each of the individual cardiac cycles in each of the arterial pressure waveforms of the first clinical dataset; identifying a systolic rise phase, a systolic decay phase, and a diastolic phase in each of the individual cardiac cycles in each of the arterial pressure waveforms of the first clinical dataset; and extracting the plurality of waveform signal measures of the first clinical dataset from each of the systolic rise phase, the systolic decay phase, and the diastolic phase from each of the individual cardiac cycles in each of the arterial pressure waveforms of the first clinical dataset.

A further embodiment of the foregoing method, wherein the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset.

A further embodiment of the foregoing method, wherein the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset.

A further embodiment of the foregoing method, wherein computing the combinatorial measures between the plurality of waveform signal measures of the first clinical dataset: performing step one by arbitrarily selecting a subset of signal measures from the plurality of waveform signal measures of the first clinical dataset; performing step two by calculating different orders of power for each signal measure of the subset of signal measures from the plurality of waveform signal measures of the first clinical dataset to generate powers of the subset of signal measures from the plurality of waveform signal measures of the first clinical dataset; performing step three by multiplying the powers of the subset of signal measures from the plurality of waveform signal measures of the first clinical dataset together to generate a product of the powers of the subset of signal measures from the plurality of waveform signal measures of the first clinical dataset; performing step four by performing receiver operating characteristic (ROC) analysis of the product of the powers of the subset of signal measures from the plurality of waveform signal measures of the first clinical dataset to arrive at a combinatorial measure for the product of the powers of the subset of signal measures from the plurality of waveform signal measures of the first clinical dataset; and repeating steps one, two, three, and four until all of the combinatorial measures have been computed between all of the plurality of waveform signal measures of the first clinical dataset.

A further embodiment of the foregoing method, wherein performing waveform analysis of the labeled arterial pressure waveforms of the second clinical dataset to calculate the plurality of waveform signal measures of the second clinical dataset comprises: identifying individual cardiac cycles in each of the arterial pressure waveforms of the second clinical dataset; identifying a dicrotic notch in each of the individual cardiac cycles in each of the arterial pressure waveforms of the second clinical dataset; identifying a systolic rise phase, a systolic decay phase, and a diastolic phase in each of the individual cardiac cycles in each of the arterial pressure waveforms of the second clinical dataset; and extracting the plurality of waveform signal measures of the second clinical dataset from each of the systolic rise phase, the systolic decay phase, and the diastolic phase from each of the individual cardiac cycles in each of the arterial pressure waveforms of the second clinical dataset.

A further embodiment of the foregoing method, wherein the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset.

A further embodiment of the foregoing method, wherein the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset.

A further embodiment of the foregoing method, wherein computing the combinatorial measures between the plurality of waveform signal measures of the second clinical dataset: performing step one by arbitrarily selecting a subset of signal measures from the plurality of waveform signal measures of the second clinical dataset; performing step two by calculating different orders of power for each of the signal measures of the subset of signal measures from the plurality of waveform signal measures of the second clinical dataset to generate powers of the subset of signal measures from the plurality of waveform signal measures of the second clinical dataset; performing step three by multiplying the powers of the subset of signal measures from the plurality of waveform signal measures of the second clinical dataset together to generate a product of the powers of the subset of signal measures from the plurality of waveform signal measures of the second clinical dataset; performing step four by performing receiver operating characteristic (ROC) analysis of the product of the powers of the subset of signal measures from the plurality of waveform signal measures of the second clinical dataset to arrive at a combinatorial measure for the product of the powers of the subset of signal measures from the plurality of waveform signal measures of the second clinical dataset; and repeating steps one, two, three, and four until all of the combinatorial measures have been computed between all of the plurality of waveform signal measures of the second clinical dataset.

A further embodiment of the foregoing method, wherein performing waveform analysis of the labeled arterial pressure waveforms of the third clinical dataset to calculate the plurality of waveform signal measures of the third clinical dataset comprises: identifying individual cardiac cycles in each of the arterial pressure waveforms of the third clinical dataset; identifying a dicrotic notch in each of the individual cardiac cycles in each of the arterial pressure waveforms of the third clinical dataset; identifying a systolic rise phase, a systolic decay phase, and a diastolic phase in each of the individual cardiac cycles in each of the arterial pressure waveforms of the third clinical dataset; and extracting the plurality of waveform signal measures of the third clinical dataset from each of the systolic rise phase, the systolic decay phase, and the diastolic phase from each of the individual cardiac cycles in each of the arterial pressure waveforms of the third clinical dataset.

A further embodiment of the foregoing method, wherein the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset.

A further embodiment of the foregoing method, wherein the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset.

A further embodiment of the foregoing method, wherein computing the combinatorial measures between the plurality of waveform signal measures of the third clinical dataset: performing step one by arbitrarily selecting a subset of signal measures from the plurality of waveform signal measures of the third clinical dataset; performing step two by calculating different orders of power for each of the signal measures of the subset of signal measures from the plurality of waveform signal measures of the third clinical dataset to generate powers of the subset of signal measures from the plurality of waveform signal measures of the third clinical dataset; performing step three by multiplying the powers of the subset of signal measures from the plurality of waveform signal measures of the third clinical dataset together to generate a product of the powers of the subset of signal measures from the plurality of waveform signal measures of the third clinical dataset; performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and repeating steps one, two, three, and four until all of the combinatorial measures have been computed between all of the plurality of waveform signal measures of the third clinical dataset.

A further embodiment of the foregoing method, wherein performing waveform analysis of the labeled arterial pressure waveforms of the fourth clinical dataset to calculate the plurality of waveform signal measures of the fourth clinical dataset comprises: identifying individual cardiac cycles in each of the arterial pressure waveforms of the fourth clinical dataset; identifying a dicrotic notch in each of the individual cardiac cycles in each of the arterial pressure waveforms of the fourth clinical dataset; identifying a systolic rise phase, a systolic decay phase, and a diastolic phase in each of the individual cardiac cycles in each of the arterial pressure waveforms of the fourth clinical dataset; and extracting the plurality of waveform signal measures of the fourth clinical dataset from each of the systolic rise phase, the systolic decay phase, and the diastolic phase from each of the individual cardiac cycles in each of the arterial pressure waveforms of the fourth clinical dataset.

A further embodiment of the foregoing method, wherein the plurality of waveform signal measures of the fourth clinical dataset correspond to hemodynamic effects from each of the systolic rise phase, the systolic decay phase, and the diastolic phase from each of the individual cardiac cycles in each of the arterial pressure waveforms of the fourth clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the fourth clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the fourth clinical dataset.

A further embodiment of the foregoing method, wherein the plurality of waveform signal measures of the fourth clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the fourth clinical dataset.

A further embodiment of the foregoing method, wherein computing the combinatorial measures between the plurality of waveform signal measures of the fourth clinical dataset: performing step one by arbitrarily selecting a subset of signal measures from the plurality of waveform signal measures of the fourth clinical dataset; performing step two by calculating different orders of power for each of the signal measures of the subset of signal measures from the plurality of waveform signal measures of the fourth clinical dataset to generate powers of the subset of signal measures from the plurality of waveform signal measures of the fourth clinical dataset; performing step three by multiplying the powers of the subset of signal measures from the plurality of waveform signal measures of the fourth clinical dataset together to generate a product of the powers of the subset of signal measures from the plurality of waveform signal measures of the fourth clinical dataset; performing step four by performing receiver operating characteristic (ROC) analysis of the product of the powers of the subset of signal measures from the plurality of waveform signal measures of the fourth clinical dataset to arrive at a combinatorial measure for the product of the powers of the subset of signal measures from the plurality of waveform signal measures of the fourth clinical dataset; and repeating steps one, two, three, and four until all of the combinatorial measures have been computed between all of the plurality of waveform signal measures of the fourth clinical dataset.

In another example, a method is disclosed for training a hemodynamic monitor to determine an aortic stenosis score of a patient. The method for training the hemodynamic monitor includes collecting a first clinical dataset containing arterial pressure waveforms from a first group of individuals with normal aortic valve function. A second clinical dataset is collected containing arterial pressure waveforms from a second group of individuals with mild aortic stenosis. Mild aortic stenosis is defined as an aortic valve area greater than 1.5 cm². The method further includes collecting a third clinical dataset containing arterial pressure waveforms from a third group of individuals with moderate aortic stenosis. Moderate aortic stenosis is defined as an aortic valve area between 1 cm² and 1.5 cm². A fourth clinical dataset is collected containing arterial pressure waveforms from a fourth group of individuals with severe aortic stenosis. Severe aortic stenosis is defined as an aortic valve area less than 1 cm². The method further includes performing waveform analysis of the arterial pressure waveforms of the first clinical dataset, the second clinical dataset, the third clinical dataset, and the fourth clinical dataset to calculate a plurality of waveform signal measures. The input features are determined by computing combinatorial measures between the plurality of waveform signal measures and selecting top signal measures from the plurality of waveform signal measures with most predictive combinatorial measures and labeling the top signal measures as the input features. The method further includes saving the input features to a memory of the hemodynamic monitor.

The method of the preceding paragraph can optionally include, additionally and/or alternatively, any one or more of the following features, configurations, and/or additional components listed below.

A further embodiment of the foregoing method, further comprising: connecting a hemodynamic sensor to the hemodynamic monitor and to the patient to input a sensed arterial pressure waveform of the patient into the hemodynamic monitor; extracting by a processor of the hemodynamic monitor values for the input features of the sensed arterial pressure waveform of the patient; determining, by the processor of the hemodynamic monitor based on the values of the input features of the sensed arterial pressure waveform, the aortic stenosis score of the patient; and outputting the aortic stenosis score of the patient to a display and/or mobile device.

A further embodiment of the foregoing method, further comprising: alerting, by the hemodynamic monitor, the patient and/or medical personnel that the aortic stenosis score is normal when the aortic stenosis score is within a first range.

A further embodiment of the foregoing method, further comprising: alerting, by the hemodynamic monitor, the patient and/or the medical personnel that the aortic stenosis score is mild when the aortic stenosis score is within a second range.

A further embodiment of the foregoing method, further comprising: alerting, by the hemodynamic monitor, the patient and/or the medical personnel that the aortic stenosis score is moderate when the aortic stenosis score is within a third range.

A further embodiment of the foregoing method, further comprising: alerting, by the hemodynamic monitor, the patient and/or the medical personnel that the aortic stenosis score is severe when the aortic stenosis score is within a fourth range.

In another example, a method is disclosed for triaging a patient for a valvular heart disorder. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The method further includes extracting, by the hemodynamic monitor, input features from the plurality of signal measures that are indicative of a valvular heart disorder score of the patient. The hemodynamic monitor determines the valvular heart disorder score of the patient based on the input features and outputs the valvular heart disorder score of the patient to a display and/or mobile device.

The method of the preceding paragraph can optionally include, additionally and/or alternatively, any one or more of the following features, configurations, and/or additional components listed below.

A further embodiment of the foregoing method, wherein the valvular heart disorder comprises at least one of aortic stenosis, mitral stenosis, mitral regurgitation, mitral valve prolapse, aortic regurgitation, and hypertrophic cardiomyopathy.

In another example, a system for triaging a patient for a valvular heart disorder includes a hemodynamic sensor that produces hemodynamic data representative of an arterial pressure waveform of the patient. The system further includes a system memory that stores valvular heart disorder software code. A user interface of the system includes a display to show a valvular heart disorder score of the patient. The system further includes a processor that is configured to execute the valvular heart disorder software code to: perform waveform analysis of the hemodynamic data to determine a plurality of signal measures; extract input features from the plurality of signal measures that are indicative of the valvular heart disorder score of the patient; determine, based on the input features, the valvular heart disorder score of the patient; and output the valvular heart disorder score of the patient to the display of the user interface.

The system of the preceding paragraph can optionally include, additionally and/or alternatively, any one or more of the following features, configurations, and/or additional components listed below.

A further embodiment of the foregoing system, wherein the valvular heart disorder comprises at least one of aortic stenosis, mitral stenosis, mitral regurgitation, mitral valve prolapse, aortic regurgitation, and hypertrophic cardiomyopathy.

In another example, a hemodynamic monitor for detecting aortic stenosis is disclosed. The hemodynamic monitor includes a non-invasive blood pressure sensor with an inflatable blood pressure bladder, a pressure controller pneumatically connected to the inflatable blood pressure bladder, and an optical transmitter and an optical receiver that are electrically connected to the pressure controller. The hemodynamic monitor further includes an integrated hardware unit with a system processor, a system memory, and a display with a user interface. The system memory includes instructions that, when executed by the system processor, are configured to adjust, by the pressure controller, a pressure within the inflatable blood pressure bladder to maintain a constant volume of an artery of a patient for a period of time based on a feedback signal generated by the optical transmitter and the optical receiver. The instructions, when executed by the system processor, are further configured to generate an arterial pressure waveform data of the patient based on the adjusted pressure within the inflatable blood pressure bladder over the period of time and extract a plurality of signal measures from the arterial pressure waveform data of the patient. The instructions, when executed by the system processor, are further configured to extract input features from the plurality of signal measures that are indicative of an aortic stenosis score of the patient; determine the aortic stenosis score of the patient based on the extracted input features; generate a first sensory alarm signal configured to generate a first sensory alert that indicates that the patient has severe aortic stenosis when the aortic stenosis score exceeds a threshold score, or generate a second sensory alarm signal configured to generate a second sensory alert that indicates that the patient does not have severe aortic stenosis when the aortic stenosis score is below the threshold score; transmit the first sensory alarm signal or the second sensory alarm signal to the user interface; and output the first sensory alert or the second sensory alert through the user interface.

The hemodynamic monitor of the preceding paragraph can optionally include, additionally and/or alternatively, any one or more of the following features, configurations, and/or additional components listed below.

A further embodiment of the foregoing hemodynamic monitor, wherein the input features of the aortic stenosis software code are determined by machine training, wherein the machine training comprises: collecting a first clinical dataset containing arterial pressure waveforms from a first group of individuals with normal aortic valve function; collecting a second clinical dataset containing arterial pressure waveforms from a second group of individuals with severe aortic stenosis, wherein severe aortic stenosis is defined as an aortic valve area less than 1 cm²;collecting a third clinical dataset containing arterial pressure waveforms from a third group of individuals with mild aortic stenosis, wherein mild aortic stenosis is defined as an aortic valve area greater than 1.5 cm²; collecting a fourth clinical dataset containing arterial pressure waveforms from a fourth group of individuals with moderate aortic stenosis, wherein moderate aortic stenosis is defined as an aortic valve area between 1 cm² and 1.5 cm²; performing waveform analysis of the arterial pressure waveforms of the first clinical dataset, the second clinical dataset, the third clinical dataset, and the fourth clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing hemodynamic monitor, wherein performing waveform analysis of the labeled arterial pressure waveforms of the first clinical dataset, the second clinical dataset, the third clinical dataset, and the fourth clinical dataset to calculate the plurality of waveform signal measures comprises: identifying individual cardiac cycles in each of the arterial pressure waveforms of the first clinical dataset, the second clinical dataset, the third clinical dataset, and the fourth clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing hemodynamic monitor, wherein the plurality of waveform signal measures corresponds to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing hemodynamic monitor, wherein the plurality of waveform signal measures comprises: a mean, a maximum, a minimum, a duration, an area, a standard deviation, derivatives, and/or morphological measures from each of the systolic rise phase, the systolic decay phase, and the diastolic phase from each of the individual cardiac cycles; and/or heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing hemodynamic monitor, wherein computing the combinatorial measures between the plurality of waveform signal measures of the first clinical dataset, the second clinical dataset, the third clinical dataset, and the fourth clinical dataset comprises: performing step one by arbitrarily selecting a subset of signal measures from the plurality of waveform signal measures; performing step two by calculating different orders of power for each signal measure of the subset of signal measures to generate powers of the subset of signal measures; performing step three by multiplying the powers of the signal measures of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing hemodynamic monitor, wherein the input features comprise a first subset and a second subset, and wherein the instructions, when executed by the system processor, are further configured to: extract the first subset and the second subset of the input features concurrently from the plurality of signal measures; concurrently determine a normal aortic stenosis score of the patient from the first subset of the input features and a severe aortic stenosis score of the patient from the second subset of the input features; and output the normal aortic stenosis score of the patient and the severe aortic stenosis score of the patient to the display of the user interface.

A further embodiment of the foregoing hemodynamic monitor, wherein the input features comprise a third subset and a fourth subset, and wherein the instructions, when executed by the system processor, are further configured to: extract the first subset, the second subset, the third subset, and the fourth subset of the input features concurrently from the plurality of signal measures; concurrently determine the normal aortic stenosis score of the patient from the first subset of the input features, the severe aortic stenosis score of the patient from the second subset of the input features, a mild aortic stenosis score of the patient from the third subset of the input features, and a moderate aortic stenosis score of the patient from the fourth subset of the input features; and output the normal aortic stenosis score of the patient, the mild aortic stenosis score of the patient, the moderate aortic stenosis score of the patient, and the severe aortic stenosis score of the patient to the display of the user interface.

In another example, a hemodynamic monitor for detecting aortic stenosis includes an arterial blood pressure sensor. The arterial pressure sensor includes a housing, a fluid input port connected via tubing to a fluid source, a catheter-side fluid port connected to a catheter inserted within an arterial system of a patient, a pressure transducer in communication with the fluid source through the fluid port, and an I/O cable in electrical communication with the pressure transducer. The hemodynamic monitor further includes an integrated hardware unit with a system processor, a system memory, a display comprising a user interface, and an analog-to-digital (ADC) converter. The system memory includes instructions that, when executed by the system processor, are configured to: receive an electrical signal from the pressure transducer over a period of time, the electrical signal based on a pressure of the arterial system of the patient transmitted through the fluid source; convert the electrical signal to a digital signal; generate an arterial pressure waveform data of the patient based on the digital signal; extract a plurality of signal measures from the arterial pressure waveform data; extract input features from the plurality of signal measures that are indicative of an aortic stenosis score of the patient; determine the aortic stenosis score of the patient based on the extracted input features; generate a first sensory alarm signal configured to generate a first sensory alert that indicates that the patient has severe aortic stenosis when the aortic stenosis score exceeds a threshold score, or generate a second sensory alarm signal configured to generate a second sensory alert that indicates that the patient does not have severe aortic stenosis when the aortic stenosis score is below the threshold score; transmit the first sensory alarm signal or the second sensory alarm signal to the user interface; and output the first sensory alert or the second sensory alert through the user interface.

The hemodynamic monitor of the preceding paragraph can optionally include, additionally and/or alternatively, any one or more of the following features, configurations, and/or additional components listed below.

A further embodiment of the foregoing hemodynamic monitor, wherein the input features of the aortic stenosis software code are determined by machine training, wherein the machine training comprises: collecting a first clinical dataset containing arterial pressure waveforms from a first group of individuals with normal aortic valve function; collecting a second clinical dataset containing arterial pressure waveforms from a second group of individuals with severe aortic stenosis, wherein severe aortic stenosis is defined as an aortic valve area less than 1 cm²;collecting a third clinical dataset containing arterial pressure waveforms from a third group of individuals with mild aortic stenosis, wherein mild aortic stenosis is defined as an aortic valve area greater than 1.5 cm²; collecting a fourth clinical dataset containing arterial pressure waveforms from a fourth group of individuals with moderate aortic stenosis, wherein moderate aortic stenosis is defined as an aortic valve area between 1 cm² and 1.5 cm²; performing waveform analysis of the arterial pressure waveforms of the first clinical dataset, the second clinical dataset, the third clinical dataset, and the fourth clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing hemodynamic monitor, wherein performing waveform analysis of the labeled arterial pressure waveforms of the first clinical dataset, the second clinical dataset, the third clinical dataset, and the fourth clinical dataset to calculate the plurality of waveform signal measures comprises: identifying individual cardiac cycles in each of the arterial pressure waveforms of the first clinical dataset, the second clinical dataset, the third clinical dataset, and the fourth clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing hemodynamic monitor, wherein the plurality of waveform signal measures corresponds to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing hemodynamic monitor, wherein the plurality of waveform signal measures comprises: a mean, a maximum, a minimum, a duration, an area, a standard deviation, derivatives, and/or morphological measures from each of the systolic rise phase, the systolic decay phase, and the diastolic phase from each of the individual cardiac cycles; and/or heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing hemodynamic monitor, wherein computing the combinatorial measures between the plurality of waveform signal measures of the first clinical dataset, the second clinical dataset, the third clinical dataset, and the fourth clinical dataset comprises: performing step one by arbitrarily selecting a subset of signal measures from the plurality of waveform signal measures; performing step two by calculating different orders of power for each signal measure of the subset of signal measures to generate powers of the subset of signal measures; performing step three by multiplying the powers of the signal measures of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing hemodynamic monitor, wherein the input features comprise a first subset and a second subset, and wherein the instructions, when executed by the system processor, are further configured to: extract the first subset and the second subset of the input features concurrently from the plurality of signal measures; concurrently determine a normal aortic stenosis score of the patient from the first subset of the input features and a severe aortic stenosis score of the patient from the second subset of the input features; and output the normal aortic stenosis score of the patient and the severe aortic stenosis score of the patient to the display of the user interface.

A further embodiment of the foregoing hemodynamic monitor, wherein the input features comprise a third subset and a fourth subset, and wherein the instructions, when executed by the system processor, are further configured to: extract the first subset, the second subset, the third subset, and the fourth subset of the input features concurrently from the plurality of signal measures; concurrently determine the normal aortic stenosis score of the patient from the first subset of the input features, the severe aortic stenosis score of the patient from the second subset of the input features, a mild aortic stenosis score of the patient from the third subset of the input features, and a moderate aortic stenosis score of the patient from the fourth subset of the input features; and output the normal aortic stenosis score of the patient, the mild aortic stenosis score of the patient, the moderate aortic stenosis score of the patient, and the severe aortic stenosis score of the patient to the display of the user interface.

In another example, a method is disclosed for triaging a patient for aortic stenosis. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The method further includes extracting, by the hemodynamic monitor, input features from the plurality of signal measures that are indicative of an aortic stenosis score of the patient. Extracting the input features includes extracting a first subset of the input features and extracting a second subset of the input features concurrently with the first subset of the input features. The hemodynamic monitor concurrently determines a normal aortic stenosis score of the patient from the first subset of the input features and a severe aortic stenosis score of the patient from the second subset of the input features. The hemodynamic monitor outputs the normal aortic stenosis score of the patient and the severe aortic stenosis score of the patient to a display and/or mobile device.

The method of the preceding paragraph can optionally include, additionally and/or alternatively, any one or more of the following features, configurations, and/or additional components listed below.

A further embodiment of the foregoing method, wherein extracting the input features further comprises: extracting a third subset of the input features concurrently with the first subset and the second subset of the input features; and extracting a fourth subset of the input features concurrently with the first subset, the second subset, and the third subset of the input features; and wherein the hemodynamic monitor concurrently determines the normal aortic stenosis score of the patient from the first subset of the input features, the severe aortic stenosis score of the patient from the second subset of the input features, a mild aortic stenosis score of the patient from the third subset of the input features, and a moderate aortic stenosis score of the patient from the fourth subset of the input features; and wherein the hemodynamic monitor outputs the normal aortic stenosis score of the patient, the mild aortic stenosis score of the patient, the moderate aortic stenosis score of the patient, and the severe aortic stenosis score of the patient to a display and/or mobile device.

A further embodiment of the foregoing method, further comprising alerting the patient and/or medical personnel that the aortic stenosis score is normal when the aortic stenosis score is within a first range; alerting the patient and/or the medical personnel that the aortic stenosis score is mild when the aortic stenosis score is within a second range; alerting the patient and/or the medical personnel that the aortic stenosis score is moderate when the aortic stenosis score is within a third range; and alerting the patient and/or the medical personnel that the aortic stenosis score is severe when the aortic stenosis score is within a fourth range.

A further embodiment of the foregoing method, further comprising: training the hemodynamic monitor for determining the aortic stenosis score of the patient, wherein training the hemodynamic monitor comprises: collecting a first clinical dataset containing arterial pressure waveforms from a first group of individuals with normal aortic valve function; labeling each of the arterial pressure waveforms of the first clinical dataset with a first label; performing waveform analysis of the labeled arterial pressure waveforms of the first clinical dataset to calculate a plurality of waveform signal measures of the first clinical dataset; determining the first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features; collecting a second clinical dataset containing arterial pressure waveforms from a second group of individuals with severe aortic stenosis, wherein severe aortic stenosis is defined as an aortic valve area less than 1 cm²; labeling each of the arterial pressure waveforms of the second clinical dataset with a second label; performing waveform analysis of the labeled arterial pressure waveforms of the second clinical dataset to calculate a plurality of waveform signal measures of the second clinical dataset; and determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features.

A further embodiment of the foregoing method, wherein training the hemodynamic monitor for determining the aortic stenosis score of the patient further comprises: collecting a third clinical dataset containing arterial pressure waveforms from a third group of individuals with mild aortic stenosis, wherein mild aortic stenosis is defined as an aortic valve area greater than 1.5 cm²; labeling each of the arterial pressure waveforms of the third clinical dataset with a third label; performing waveform analysis of the labeled arterial pressure waveforms of the third clinical dataset to calculate a plurality of waveform signal measures of the third clinical dataset; determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features; collecting a fourth clinical dataset containing arterial pressure waveforms from a fourth group of individuals with moderate aortic stenosis, wherein moderate aortic stenosis is defined as an aortic valve area between 1 cm² and 1.5 cm²; labeling each of the arterial pressure waveforms of the fourth clinical dataset with a fourth label; performing waveform analysis of the labeled arterial pressure waveforms of the fourth clinical dataset to calculate a plurality of waveform signal measures of the fourth clinical dataset; and determining a fourth subset of the input features by computing combinatorial measures between the plurality of waveform signal measures of the fourth clinical dataset and selecting top signal measures from the plurality of waveform signal measures of the fourth clinical dataset with most predictive combinatorial measures and labeling the top signal measures of the fourth clinical data set as the fourth subset of the input features.

A further embodiment of the foregoing method, wherein: performing waveform analysis of the labeled arterial pressure waveforms of the first clinical dataset to calculate the plurality of waveform signal measures of the first clinical dataset comprises: identifying individual cardiac cycles in each of the arterial pressure waveforms of the first clinical dataset; identifying a dicrotic notch in each of the individual cardiac cycles in each of the arterial pressure waveforms of the first clinical dataset; identifying a systolic rise phase, a systolic decay phase, and a diastolic phase in each of the individual cardiac cycles in each of the arterial pressure waveforms of the first clinical dataset; and extracting the plurality of waveform signal measures of the first clinical dataset from each of the systolic rise phase, the systolic decay phase, and the diastolic phase from each of the individual cardiac cycles in each of the arterial pressure waveforms of the first clinical dataset; performing waveform analysis of the labeled arterial pressure waveforms of the second clinical dataset to calculate the plurality of waveform signal measures of the second clinical dataset comprises: identifying individual cardiac cycles in each of the arterial pressure waveforms of the second clinical dataset; identifying a dicrotic notch in each of the individual cardiac cycles in each of the arterial pressure waveforms of the second clinical dataset; identifying a systolic rise phase, a systolic decay phase, and a diastolic phase in each of the individual cardiac cycles in each of the arterial pressure waveforms of the second clinical dataset; and extracting the plurality of waveform signal measures of the second clinical dataset from each of the systolic rise phase, the systolic decay phase, and the diastolic phase from each of the individual cardiac cycles in each of the arterial pressure waveforms of the second clinical dataset; performing waveform analysis of the labeled arterial pressure waveforms of the third clinical dataset to calculate the plurality of waveform signal measures of the third clinical dataset comprises: identifying individual cardiac cycles in each of the arterial pressure waveforms of the third clinical dataset; identifying a dicrotic notch in each of the individual cardiac cycles in each of the arterial pressure waveforms of the third clinical dataset; identifying a systolic rise phase, a systolic decay phase, and a diastolic phase in each of the individual cardiac cycles in each of the arterial pressure waveforms of the third clinical dataset; and extracting the plurality of waveform signal measures of the third clinical dataset from each of the systolic rise phase, the systolic decay phase, and the diastolic phase from each of the individual cardiac cycles in each of the arterial pressure waveforms of the third clinical dataset; and performing waveform analysis of the labeled arterial pressure waveforms of the fourth clinical dataset to calculate the plurality of waveform signal measures of the fourth clinical dataset comprises: identifying individual cardiac cycles in each of the arterial pressure waveforms of the fourth clinical dataset; identifying a dicrotic notch in each of the individual cardiac cycles in each of the arterial pressure waveforms of the fourth clinical dataset; identifying a systolic rise phase, a systolic decay phase, and a diastolic phase in each of the individual cardiac cycles in each of the arterial pressure waveforms of the fourth clinical dataset; and extracting the plurality of waveform signal measures of the fourth clinical dataset from each of the systolic rise phase, the systolic decay phase, and the diastolic phase from each of the individual cardiac cycles in each of the arterial pressure waveforms of the fourth clinical dataset.

A further embodiment of the foregoing method, wherein: the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle; the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle; the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle; and/or the plurality of waveform signal measures of the fourth clinical dataset correspond to hemodynamic effects from each of the systolic rise phase, the systolic decay phase, and the diastolic phase from each of the individual cardiac cycles in each of the arterial pressure waveforms of the fourth clinical dataset, and wherein the hemodynamic effects comprise contractility, aortic compliance, stroke volume, vascular tone, afterload, and full cardiac cycle.

A further embodiment of the foregoing method, wherein: the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset; the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset; the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset; and/or the plurality of waveform signal measures of the fourth clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the fourth clinical dataset.

A further embodiment of the foregoing method, wherein: the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset; the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset; the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset; and/or the plurality of waveform signal measures of the fourth clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the fourth clinical dataset.

A further embodiment of the foregoing method, wherein computing the combinatorial measures between the plurality of waveform signal measures of the first clinical dataset comprises: performing step one by arbitrarily selecting a subset of signal measures from the plurality of waveform signal measures of the first clinical dataset; performing step two by calculating different orders of power for each signal measure of the subset of signal measures from the plurality of waveform signal measures of the first clinical dataset to generate powers of the subset of signal measures from the plurality of waveform signal measures of the first clinical dataset; performing step three by multiplying the powers of the subset of signal measures from the plurality of waveform signal measures of the first clinical dataset together to generate a product of the powers of the subset of signal measures from the plurality of waveform signal measures of the first clinical dataset; performing step four by performing receiver operating characteristic (ROC) analysis of the product of the powers of the subset of signal measures from the plurality of waveform signal measures of the first clinical dataset to arrive at a combinatorial measure for the product of the powers of the subset of signal measures from the plurality of waveform signal measures of the first clinical dataset; and repeating steps one, two, three, and four until all of the combinatorial measures have been computed between all of the plurality of waveform signal measures of the first clinical dataset.

A further embodiment of the foregoing method, wherein computing the combinatorial measures between the plurality of waveform signal measures of the second clinical dataset comprises: performing step one by arbitrarily selecting a subset of signal measures from the plurality of waveform signal measures of the second clinical dataset; performing step two by calculating different orders of power for each of the signal measures of the subset of signal measures from the plurality of waveform signal measures of the second clinical dataset to generate powers of the subset of signal measures from the plurality of waveform signal measures of the second clinical dataset; performing step three by multiplying the powers of the subset of signal measures from the plurality of waveform signal measures of the second clinical dataset together to generate a product of the powers of the subset of signal measures from the plurality of waveform signal measures of the second clinical dataset; performing step four by performing receiver operating characteristic (ROC) analysis of the product of the powers of the subset of signal measures from the plurality of waveform signal measures of the second clinical dataset to arrive at a combinatorial measure for the product of the powers of the subset of signal measures from the plurality of waveform signal measures of the second clinical dataset; and repeating steps one, two, three, and four until all of the combinatorial measures have been computed between all of the plurality of waveform signal measures of the second clinical dataset.

A further embodiment of the foregoing method, wherein computing the combinatorial measures between the plurality of waveform signal measures of the third clinical dataset comprises: performing step one by arbitrarily selecting a subset of signal measures from the plurality of waveform signal measures of the third clinical dataset; performing step two by calculating different orders of power for each of the signal measures of the subset of signal measures from the plurality of waveform signal measures of the third clinical dataset to generate powers of the subset of signal measures from the plurality of waveform signal measures of the third clinical dataset; performing step three by multiplying the powers of the subset of signal measures from the plurality of waveform signal measures of the third clinical dataset together to generate a product of the powers of the subset of signal measures from the plurality of waveform signal measures of the third clinical dataset; performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and repeating steps one, two, three, and four until all of the combinatorial measures have been computed between all of the plurality of waveform signal measures of the third clinical dataset; and wherein computing the combinatorial measures between the plurality of waveform signal measures of the fourth clinical dataset: performing step one by arbitrarily selecting a subset of signal measures from the plurality of waveform signal measures of the fourth clinical dataset; performing step two by calculating different orders of power for each of the signal measures of the subset of signal measures from the plurality of waveform signal measures of the fourth clinical dataset to generate powers of the subset of signal measures from the plurality of waveform signal measures of the fourth clinical dataset; performing step three by multiplying the powers of the subset of signal measures from the plurality of waveform signal measures of the fourth clinical dataset together to generate a product of the powers of the subset of signal measures from the plurality of waveform signal measures of the fourth clinical dataset; performing step four by performing receiver operating characteristic (ROC) analysis of the product of the powers of the subset of signal measures from the plurality of waveform signal measures of the fourth clinical dataset to arrive at a combinatorial measure for the product of the powers of the subset of signal measures from the plurality of waveform signal measures of the fourth clinical dataset; and repeating steps one, two, three, and four until all of the combinatorial measures have been computed between all of the plurality of waveform signal measures of the fourth clinical dataset.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A hemodynamic monitor for detecting aortic stenosis, the hemodynamic monitor comprising:
a non-invasive blood pressure sensor comprising an inflatable blood pressure bladder, a pressure controller pneumatically connected to the inflatable blood pressure bladder, and an optical transmitter and an optical receiver that are electrically connected to the pressure controller;
an integrated hardware unit comprising:
a system processor;
a system memory;
a display comprising a user interface; and
wherein the system memory comprises instructions that, when executed by the system processor, are configured to:
adjust, by the pressure controller, a pressure within the inflatable blood pressure bladder to maintain a constant volume of an artery of a patient for a period of time based on a feedback signal generated by the optical transmitter and the optical receiver;
generate an arterial pressure waveform data of the patient based on the adjusted pressure within the inflatable blood pressure bladder over the period of time;
extract a plurality of signal measures from the arterial pressure waveform data of the patient;
extract input features from the plurality of signal measures that are indicative of an aortic stenosis score of the patient;
determine the aortic stenosis score of the patient based on the extracted input features;
generate a first sensory alarm signal configured to generate a first sensory alert that indicates that the patient has severe aortic stenosis when the aortic stenosis score exceeds a threshold score, or generate a second sensory alarm signal configured to generate a second sensory alert that indicates that the patient does not have severe aortic stenosis when the aortic stenosis score is below the threshold score;
transmit the first sensory alarm signal or the second sensory alarm signal to the user interface; and
output the first sensory alert or the second sensory alert through the user interface.

2. The hemodynamic monitor of claim 1, wherein the input features are determined by machine training, wherein the machine training comprises:
collecting a first clinical dataset containing arterial pressure waveforms from a first group of individuals with normal aortic valve function;
collecting a second clinical dataset containing arterial pressure waveforms from a second group of individuals with severe aortic stenosis, wherein severe aortic stenosis is defined as an aortic valve area less than 1 cm²;
collecting a third clinical dataset containing arterial pressure waveforms from a third group of individuals with mild aortic stenosis, wherein mild aortic stenosis is defined as an aortic valve area greater than 1.5 cm²;
collecting a fourth clinical dataset containing arterial pressure waveforms from a fourth group of individuals with moderate aortic stenosis, wherein moderate aortic stenosis is defined as an aortic valve area between 1 cm² and 1.5 cm²;
performing waveform analysis of the arterial pressure waveforms of the first clinical dataset, the second clinical dataset, the third clinical dataset, and the fourth clinical dataset to calculate a plurality of waveform signal measures; and
determining the input features by computing combinatorial measures between the plurality of waveform signal measures and selecting top signal measures from the plurality of waveform signal measures with most predictive combinatorial measures and labeling the top signal measures as the input features.

3. The hemodynamic monitor of claim 2, wherein performing waveform analysis of the arterial pressure waveforms of the first clinical dataset, the second clinical dataset, the third clinical dataset, and the fourth clinical dataset to calculate the plurality of waveform signal measures comprises:
identifying individual cardiac cycles in each of the arterial pressure waveforms of the first clinical dataset, the second clinical dataset, the third clinical dataset, and the fourth clinical dataset;
identifying a dicrotic notch in each of the individual cardiac cycles;
identifying a systolic rise phase, a systolic decay phase, and a diastolic phase in each of the individual cardiac cycles; and
extracting the plurality of waveform signal measures from each of the systolic rise phase, the systolic decay phase, and the diastolic phase from each of the individual cardiac cycles.

4. The hemodynamic monitor of claim 3, wherein the plurality of waveform signal measures corresponds to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

5. The hemodynamic monitor of claim 4, wherein the plurality of waveform signal measures comprises:
a mean, a maximum, a minimum, a duration, an area, a standard deviation, derivatives, and/or morphological measures from each of the systolic rise phase, the systolic decay phase, and the diastolic phase from each of the individual cardiac cycles; and/or
heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

6. The hemodynamic monitor of claim 5, wherein computing the combinatorial measures between the plurality of waveform signal measures of the first clinical dataset, the second clinical dataset, the third clinical dataset, and the fourth clinical dataset comprises:
performing step one by arbitrarily selecting a subset of signal measures from the plurality of waveform signal measures;
performing step two by calculating different orders of power for each signal measure of the subset of signal measures to generate powers of the subset of signal measures;
performing step three by multiplying the powers of the signal measures of the subset of signal measures together to generate the product of the powers of the subset of signal measures;
performing step four by performing receiver operating characteristic (ROC) analysis of the product to arrive at a combinatorial measure for the subset of signal measures; and
repeating steps one, two, three, and four until all of the combinatorial measures have been computed between all of the plurality of waveform signal measures.

7. The hemodynamic monitor of claim 6, wherein the input features comprise a first subset and a second subset, and wherein the instructions, when executed by the system processor, are further configured to:
extract the first subset and the second subset of the input features concurrently from the plurality of signal measures;
concurrently determine a normal aortic stenosis score of the patient from the first subset of the input features and a severe aortic stenosis score of the patient from the second subset of the input features; and
output the normal aortic stenosis and the severe aortic stenosis score of the patient to the display of the user interface.

8. The hemodynamic monitor of claim 7, wherein the input features comprise a third subset and a fourth subset, and wherein the instructions, when executed by the system processor, are further configured to:
extract the first subset, the second subset, the third subset, and the fourth subset of the input features concurrently from the plurality of signal measures;
concurrently determine the normal aortic stenosis score of the patient from the first subset of the input features, the severe aortic stenosis score of the patient from the second subset of the input features, a mild aortic stenosis score of the patient from the third subset of the input features, and a moderate aortic stenosis score of the patient from the fourth subset of the input features;
output the normal aortic stenosis score of the patient, the mild aortic stenosis score of the patient, the moderate aortic stenosis score of the patient, and the severe aortic stenosis score of the patient to the display of the user interface.

9. A method for triaging a patient for aortic stenosis, the method comprising:
receiving, by a hemodynamic monitor, sensed hemodynamic data representative of an arterial pressure waveform of the patient;
performing, by the hemodynamic monitor, waveform analysis of the sensed hemodynamic data to calculate a plurality of signal measures of the sensed hemodynamic data;
extracting, by the hemodynamic monitor, input features from the plurality of signal measures that are indicative of an aortic stenosis score of the patient, wherein
extracting the input features comprises:
extracting a first subset of the input features; and
extracting a second subset of the input features concurrently with the first subset of the input features;
concurrently determining, by the hemodynamic monitor, a normal aortic stenosis score of the patient from the first subset of the input features and a severe aortic stenosis score of the patient from the second subset of the input features; and
outputting the normal aortic stenosis score and the severe aortic stenosis score of the patient to a display and/or mobile device.

10. The method of claim 9, wherein extracting the input features further comprises:
extracting a third subset of the input features concurrently with the first subset and the second subset of the input features; and
extracting a fourth subset of the input features concurrently with the first subset, the second subset, and the third subset of the input features; and
wherein the hemodynamic monitor concurrently determines the normal aortic stenosis score of the patient from the first subset of the input features, the severe aortic stenosis score of the patient from the second subset of the input features, a mild aortic stenosis score of the patient from the third subset of the input features, and a moderate aortic stenosis score of the patient from the fourth subset of the input features; and
wherein the hemodynamic monitor outputs the normal aortic stenosis score of the patient, the mild aortic stenosis score of the patient, the moderate aortic stenosis score of the patient, and the severe aortic stenosis score of the patient to a display and/or mobile device.

11. The method of claim 10, further comprising
alerting the patient and/or medical personnel that the aortic stenosis score is normal when the aortic stenosis score is within a first range;
alerting the patient and/or the medical personnel that the aortic stenosis score is mild when the aortic stenosis score is within a second range;
alerting the patient and/or the medical personnel that the aortic stenosis score is moderate when the aortic stenosis score is within a third range; and
alerting the patient and/or the medical personnel that the aortic stenosis score is severe when the aortic stenosis score is within a fourth range.

12. The method of claim 11, further comprising:
training the hemodynamic monitor for determining the aortic stenosis score of the patient, wherein training the hemodynamic monitor comprises:
collecting a first clinical dataset containing arterial pressure waveforms from a first group of individuals with normal aortic valve function;
labeling each of the arterial pressure waveforms of the first clinical dataset with a first label;
performing waveform analysis of the labeled arterial pressure waveforms of the first clinical dataset to calculate a plurality of waveform signal measures of the first clinical dataset;
determining the first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features;
collecting a second clinical dataset containing arterial pressure waveforms from a second group of individuals with severe aortic stenosis, wherein severe aortic stenosis is defined as an aortic valve area less than 1 cm²;
labeling each of the arterial pressure waveforms of the second clinical dataset with a second label;
performing waveform analysis of the labeled arterial pressure waveforms of the second clinical dataset to calculate a plurality of waveform signal measures of the second clinical dataset; and
determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features.

13. The method of claim 12, wherein training the hemodynamic monitor for determining the aortic stenosis score of the patient further comprises:
collecting a third clinical dataset containing arterial pressure waveforms from a third group of individuals with mild aortic stenosis, wherein mild aortic stenosis is defined as an aortic valve area greater than 1.5 cm²;
labeling each of the arterial pressure waveforms of the third clinical dataset with a third label;
performing waveform analysis of the labeled arterial pressure waveforms of the third clinical dataset to calculate a plurality of waveform signal measures of the third clinical dataset;
determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features;
collecting a fourth clinical dataset containing arterial pressure waveforms from a fourth group of individuals with moderate aortic stenosis, wherein moderate aortic stenosis is defined as an aortic valve area between 1 cm² and 1.5 cm²;
labeling each of the arterial pressure waveforms of the fourth clinical dataset with a fourth label;
performing waveform analysis of the labeled arterial pressure waveforms of the fourth clinical dataset to calculate a plurality of waveform signal measures of the fourth clinical dataset; and
determining a fourth subset of the input features by computing combinatorial measures between the plurality of waveform signal measures of the fourth clinical dataset and selecting top signal measures from the plurality of waveform signal measures of the fourth clinical dataset with most predictive combinatorial measures and labeling the top signal measures of the fourth clinical data set as the fourth subset of the input features.

14. The method of claim 13, wherein:
performing waveform analysis of the labeled arterial pressure waveforms of the first clinical dataset to calculate the plurality of waveform signal measures of the first clinical dataset comprises:
identifying individual cardiac cycles in each of the arterial pressure waveforms of the first clinical dataset;
identifying a dicrotic notch in each of the individual cardiac cycles in each of the arterial pressure waveforms of the first clinical dataset;
identifying a systolic rise phase, a systolic decay phase, and a diastolic phase in each of the individual cardiac cycles in each of the arterial pressure waveforms of the first clinical dataset; and
extracting the plurality of waveform signal measures of the first clinical dataset from each of the systolic rise phase, the systolic decay phase, and the diastolic phase from each of the individual cardiac cycles in each of the arterial pressure waveforms of the first clinical dataset;
performing waveform analysis of the labeled arterial pressure waveforms of the second clinical dataset to calculate the plurality of waveform signal measures of the second clinical dataset comprises:
identifying individual cardiac cycles in each of the arterial pressure waveforms of the second clinical dataset;
identifying a dicrotic notch in each of the individual cardiac cycles in each of the arterial pressure waveforms of the second clinical dataset;
identifying a systolic rise phase, a systolic decay phase, and a diastolic phase in each of the individual cardiac cycles in each of the arterial pressure waveforms of the second clinical dataset; and
extracting the plurality of waveform signal measures of the second clinical dataset from each of the systolic rise phase, the systolic decay phase, and the diastolic phase from each of the individual cardiac cycles in each of the arterial pressure waveforms of the second clinical dataset;
performing waveform analysis of the labeled arterial pressure waveforms of the third clinical dataset to calculate the plurality of waveform signal measures of the third clinical dataset comprises:
identifying individual cardiac cycles in each of the arterial pressure waveforms of the third clinical dataset;
identifying a dicrotic notch in each of the individual cardiac cycles in each of the arterial pressure waveforms of the third clinical dataset;
identifying a systolic rise phase, a systolic decay phase, and a diastolic phase in each of the individual cardiac cycles in each of the arterial pressure waveforms of the third clinical dataset; and
extracting the plurality of waveform signal measures of the third clinical dataset from each of the systolic rise phase, the systolic decay phase, and the diastolic phase from each of the individual cardiac cycles in each of the arterial pressure waveforms of the third clinical dataset; and
performing waveform analysis of the labeled arterial pressure waveforms of the fourth clinical dataset to calculate the plurality of waveform signal measures of the fourth clinical dataset comprises:
identifying individual cardiac cycles in each of the arterial pressure waveforms of the fourth clinical dataset;
identifying a dicrotic notch in each of the individual cardiac cycles in each of the arterial pressure waveforms of the fourth clinical dataset;
identifying a systolic rise phase, a systolic decay phase, and a diastolic phase in each of the individual cardiac cycles in each of the arterial pressure waveforms of the fourth clinical dataset; and
extracting the plurality of waveform signal measures of the fourth clinical dataset from each of the systolic rise phase, the systolic decay phase, and the diastolic phase from each of the individual cardiac cycles in each of the arterial pressure waveforms of the fourth clinical dataset.

15. The method of claim 14, wherein:
the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle;
the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle;
the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle; and/or
the plurality of waveform signal measures of the fourth clinical dataset correspond to hemodynamic effects from each of the systolic rise phase, the systolic decay phase, and the diastolic phase from each of the individual cardiac cycles in each of the arterial pressure waveforms of the fourth clinical dataset, and wherein the hemodynamic effects comprise contractility, aortic compliance, stroke volume, vascular tone, afterload, and full cardiac cycle.

16. The method of claim 15, wherein:
the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset;
the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset;
the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset; and/or
the plurality of waveform signal measures of the fourth clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the fourth clinical dataset.

17. The method of claim 16, wherein:
the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset;
the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset;
the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset; and/or
the plurality of waveform signal measures of the fourth clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the fourth clinical dataset.

18. The method of claim 17, wherein computing the combinatorial measures between the plurality of waveform signal measures of the first clinical dataset comprises:
performing step one by arbitrarily selecting a subset of signal measures from the plurality of waveform signal measures of the first clinical dataset;
performing step two by calculating different orders of power for each signal measure of the subset of signal measures from the plurality of waveform signal measures of the first clinical dataset to generate powers of the subset of signal measures from the plurality of waveform signal measures of the first clinical dataset;
performing step three by multiplying the powers of the subset of signal measures from the plurality of waveform signal measures of the first clinical dataset together to generate a product of the powers of the subset of signal measures from the plurality of waveform signal measures of the first clinical dataset;
performing step four by performing receiver operating characteristic (ROC) analysis of the product of the powers of the subset of signal measures from the plurality of waveform signal measures of the first clinical dataset to arrive at a combinatorial measure for the product of the powers of the subset of signal measures from the plurality of waveform signal measures of the first clinical dataset; and
repeating steps one, two, three, and four until all of the combinatorial measures have been computed between all of the plurality of waveform signal measures of the first clinical dataset.

19. The method of claim 18, wherein computing the combinatorial measures between the plurality of waveform signal measures of the second clinical dataset comprises:
performing step one by arbitrarily selecting a subset of signal measures from the plurality of waveform signal measures of the second clinical dataset;
performing step two by calculating different orders of power for each of the signal measures of the subset of signal measures from the plurality of waveform signal measures of the second clinical dataset to generate powers of the subset of signal measures from the plurality of waveform signal measures of the second clinical dataset;
performing step three by multiplying the powers of the subset of signal measures from the plurality of waveform signal measures of the second clinical dataset together to generate a product of the powers of the subset of signal measures from the plurality of waveform signal measures of the second clinical dataset;
performing step four by performing receiver operating characteristic (ROC) analysis of the product of the powers of the subset of signal measures from the plurality of waveform signal measures of the second clinical dataset to arrive at a combinatorial measure for the product of the powers of the subset of signal measures from the plurality of waveform signal measures of the second clinical dataset; and
repeating steps one, two, three, and four until all of the combinatorial measures have been computed between all of the plurality of waveform signal measures of the second clinical dataset.

20. The method of claim 19, wherein computing the combinatorial measures between the plurality of waveform signal measures of the third clinical dataset comprises:
performing step one by arbitrarily selecting a subset of signal measures from the plurality of waveform signal measures of the third clinical dataset;
performing step two by calculating different orders of power for each of the signal measures of the subset of signal measures from the plurality of waveform signal measures of the third clinical dataset to generate powers of the subset of signal measures from the plurality of waveform signal measures of the third clinical dataset;
performing step three by multiplying the powers of the subset of signal measures from the plurality of waveform signal measures of the third clinical dataset together to generate a product of the powers of the subset of signal measures from the plurality of waveform signal measures of the third clinical dataset;
performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and
repeating steps one, two, three, and four until all of the combinatorial measures have been computed between all of the plurality of waveform signal measures of the third clinical dataset; and
wherein computing the combinatorial measures between the plurality of waveform signal measures of the fourth clinical dataset:
performing step one by arbitrarily selecting a subset of signal measures from the plurality of waveform signal measures of the fourth clinical dataset;
performing step two by calculating different orders of power for each of the signal measures of the subset of signal measures from the plurality of waveform signal measures of the fourth clinical dataset to generate powers of the subset of signal measures from the plurality of waveform signal measures of the fourth clinical dataset;
performing step three by multiplying the powers of the subset of signal measures from the plurality of waveform signal measures of the fourth clinical dataset together to generate a product of the powers of the subset of signal measures from the plurality of waveform signal measures of the fourth clinical dataset;
performing step four by performing receiver operating characteristic (ROC) analysis of the product of the powers of the subset of signal measures from the plurality of waveform signal measures of the fourth clinical dataset to arrive at a combinatorial measure for the product of the powers of the subset of signal measures from the plurality of waveform signal measures of the fourth clinical dataset; and
repeating steps one, two, three, and four until all of the combinatorial measures have been computed between all of the plurality of waveform signal measures of the fourth clinical dataset.
